(19) United States
(12) Patent Application Publication
(10) Pub. No.: US 2024/0293816 A1
(43) Pub. Date: Sep. 5, 2024

(54) DEVICE HAVING HORIZONTAL NANOCHANNEL FOR NANOPORE SEQUENCING

(71) Applicant: ILLUMINA, INC., San Diego, CA (US)

(72) Inventors: Rean Silke Musa, La Jolla, CA (US); Anthony Flannery, Bainbridge Island, WA (US); Boyan Boyanov, San Diego, CA (US); Nigel COBURN, San Diego, CA (US); Sharis MINASSIAN, San Diego, CA (US)

(21) Appl. No.: 18/574,214

(22) PCT Filed: Jun. 30, 2022
(86) PCT No.: PCT/US2022/035837

§ 371 Date: Dec. 26, 2023

Related U.S. Application Data
(60) Provisional application No. 63/202,971, filed on Jul. 1, 2021.

(51) Int. Cl. B01L 3/00; C12Q 1/6869

(52) U.S. Cl. CPC B01L 3/502761; C12Q 1/6869; B01L 2300/0645; B01L 2300/0829; B01L 2300/0893; B01L 2400/0427; B01L 2400/046

(57) ABSTRACT

Devices for sequencing biopolymers, methods of manufacturing the devices, and methods of using the devices are disclosed. In one example, such a device has a nanopore and a horizontal nanochannel. In some embodiments, the horizontal nanochannel may take a tortuous path. In some embodiments, such a device includes gas or air bubble generators or pressure pulse generators to block or unblock the horizontal nanochannel.

### BACKGROUND

Some polynucleotide sequencing techniques involve performing a large number of controlled reactions on support surfaces or within predefined reaction chambers. The controlled reactions may then be observed or detected, and subsequent analysis may help identify properties of the polynucleotide involved in the reaction. Examples of such sequencing techniques include next-generation sequencing or massive parallel sequencing involving sequencing-by-ligation, sequencing-by-synthesis, reversible terminator chemistry, or pyrosequencing approaches.

Some polynucleotide sequencing techniques utilize a nanopore, which can provide a path for an ionic electrical current. For example, as the polynucleotide traverses through the nanopore, it influences the ionic current through the nanopore. Each passing nucleotide, or series of nucleotides, that passes through the nanopore yields a characteristic electrical current. These characteristic electrical currents as a result of the traversing polynucleotide can be recorded to determine the sequence of the polynucleotide.

### SUMMARY

Provided in examples herein are devices for sequencing biopolymers, e.g., polynucleotides, proteins, or peptides, methods of manufacturing the devices, and methods of using the devices.

In some embodiments, a nanopore sequencing device is disclosed. In some embodiments, the nanopore sequencing device comprises a substrate comprising a dielectric layer and at least one sensing electrode on a surface of the dielectric layer; a cis well associated with a cis electrode; a trans well associated with a trans electrode; a middle well associated with the sensing electrode and positioned on the substrate, wherein the middle well is positioned on the substrate and in fluid communication with the cis well and the trans well; a nanopore fluidically connecting the cis well and the middle well; and a nanochannel fluidically connecting the middle well and the trans well, wherein the nanochannel is formed on the surface of the substrate.

The nanopore sequencing device of any of the preceding embodiments, wherein the nanochannel does not comprise a through-hole in the substrate.

The nanopore sequencing device of any of the preceding embodiments, wherein the nanopore is positioned in and through a membrane separating the cis well and the middle well.

The nanopore sequencing device of any of the preceding embodiments, wherein the membrane is formed of lipid, silicon, graphene, a solid-state material, a synthetic material, a biomimetic equivalent of lipid, or any combination thereof.

The nanopore sequencing device of any of the preceding embodiments, wherein the nanopore is a hollow in a structure formed of one or more polynucleotides, one or more polypeptides, one or more types of biopolymers, one or more carbon nanotubes, one or more types of solid-state materials, or any combination thereof disposed in the membrane.

The nanopore sequencing device of any of the preceding embodiments, wherein the nanopore comprises biologically derived material.

The nanopore sequencing device of any of the preceding embodiments, wherein the nanopore comprises a porin.

The nanopore sequencing device of any of the preceding embodiments, wherein the nanopore comprises non-biologically derived material.

The nanopore sequencing device of any of the preceding embodiments, wherein at least the cis well or the trans well is positioned horizontally side-by-side with the middle well.

The nanopore sequencing device of any of the preceding embodiments, wherein both the cis well and the trans well are positioned horizontally side-by-side with the middle well.

The nanopore sequencing device of any of the preceding embodiments, wherein the cis well is positioned horizontally side-by-side with the middle well, and the trans well is positioned vertically adjacent to the middle well.

The nanopore sequencing device of any of the preceding embodiments, wherein the trans well is positioned horizontally side-by-side with the middle well, and the cis well is positioned vertically adjacent to the middle well.

The nanopore sequencing device of any of the preceding embodiments, wherein the middle well has a characteristic width of about 5 μm to about 200 μm.

The nanopore sequencing device of any of the preceding embodiments, wherein the middle well has a characteristic depth of about 5 μm to about 200 μm.

The nanopore sequencing device of any of the preceding embodiments, wherein the cis well has a characteristic width of about 10 μm to about 10 mm.

The nanopore sequencing device of any of the preceding embodiments, wherein the trans well has a characteristic size of about 10 μm to about 10 mm.

The nanopore sequencing device of any of the preceding embodiments, wherein the nanochannel has a tortuous path.

The nanopore sequencing device of any of the preceding embodiments, wherein the tortuous path comprises a rectangular wave shape, a sine wave shape, a sawtooth shape, a zigzag shape, a spiral shape, or any combination thereof.

The nanopore sequencing device of any of the preceding embodiments, wherein the nanochannel has a path length that is chosen to achieve a desired fluidic, ionic, and/or electrical resistance.

The nanopore sequencing device of any of the preceding embodiments, wherein the nanochannel is about 5 nm to about 200 nm wide.

The nanopore sequencing device of any of the preceding embodiments, wherein the nanochannel has a footprint with a length of between about 5 μm and about 500 μm.

The nanopore sequencing device of any of the preceding embodiments, wherein the path length of the nanochannel is about 1.5 to about 50 times the length of the nanochannel footprint.

The nanopore sequencing device of any of the preceding embodiments, further comprising at least one bubble generator, at least one pressure pulse generator, or any combination thereof to control a liquid flow in at least one of the second nanoscale openings.

The nanopore sequencing device of any of the preceding embodiments, further comprising: a plurality of middle wells, wherein each middle well is associated with a respective sensing electrode; each middle well is in fluid communication with the cis well through a respective nanopore; and each middle well is in fluid communication with the trans well through a respective nanochannel, wherein the respective nanochannel is oriented parallel to the substrate surface.

The nanopore sequencing device of any of the preceding embodiments, wherein the respective nanopore is positioned in and through a respective membrane separating each of the middle wells and the cis well.

The nanopore sequencing device of any of the preceding embodiments, wherein the trans well is a common trans channel in fluid communication with the plurality of middle wells through respective nanochannels.

The nanopore sequencing device of any of the preceding embodiments, wherein the cis well is a common cis channel in fluid communication with the plurality of middle wells through respective nanopores.

The nanopore sequencing device of any of the preceding embodiments, wherein the middle wells are arranged in an ordered array.

The nanopore sequencing device of any of the preceding embodiments, wherein the device comprises at least 1,000,000 middle wells.

The nanopore sequencing device of any of the preceding embodiments, wherein the device further comprises a gas bubble generator configured to generate a gas bubble to modulate or block a flow of current, ions, and/or fluid in the respective nanochannel.

The nanopore sequencing device of claim 29, wherein the gas bubble generator comprises the respective sensing electrode configured to generate the gas bubble vis electrolysis.

The nanopore sequencing device of any of the preceding embodiments, wherein the gas bubble generator comprises an electrode on the bottom of the nanochannel configured to generate the gas bubble via electrolysis or electrode wetting.

The nanopore sequencing device of any of the preceding embodiments, wherein the gas bubble generator comprises a resistive heater underneath the nanochannel configured to generate the gas bubble.

The nanopore sequencing device of any of the preceding embodiments, further comprises a gas bubble annihilator.

The nanopore sequencing device of any of the preceding embodiments, wherein the gas bubble annihilator comprises an actuator or a piezoelectric element.

In some embodiments, a method of manufacturing the nanopore sequencing device is disclosed. In some embodiments, the method comprises: providing a first substrate comprising a dielectric layer and at least one sensing electrode on a surface of the first substrate; forming at least one nanochannel on the surface of the first substrate; and forming a first patterned layer over the substrate, wherein the first patterned layer comprises a trans well adjacent to the at least one nanochannel and at least one middle well above the sensing electrode.

The method of any of the previous embodiments, wherein the at least one nanochannel is formed along the surface of the first substrate without forming a through-hole in the substrate.

The method of any of the previous embodiments, wherein forming at least one nanochannel comprises etching the nanochannel into the surface of the first substrate.

The method of any of the previous embodiments, wherein forming at least one nanochannel comprises forming a patterned nanochannel structure on the surface of the first substrate.

The method of any of the previous embodiments, wherein forming a first patterned layer comprises depositing a patterning material layer over the substrate, and patterning the patterning material layer to expose the at least one sensing electrode and openings to the at least one nanochannel.

The method of any of the previous embodiments, further comprising forming an oxide or nitride layer in the at least one nanochannel, thereby reducing the width of the nanochannel.

The method of any of the previous embodiments, further comprising: depositing a capping layer over the first substrate prior to forming the first patterned layer; and patterning the capping layer to expose the at least one sensing electrode and openings to the at least one nanochannel.

The method of any of the previous embodiments, wherein the trans well and the middle well are positioned side-by-side on the first substrate.

The method of any of the previous embodiments, wherein the first patterned layer further comprises a cis well next to and positioned side-by-side to the at least one middle well.

The method of any of the previous embodiments, further comprising: providing a second substrate have a second patterned layer attached; and bonding the second patterned layer with the first patterned layer, thereby further define the cis well, the middle well, and the trans well between the first substrate and the second substrate.

The method of any of the previous embodiments, wherein the second substrate further comprises fluidic inlet and/or outlet holes.

The method of any of the previous embodiments, further comprising introducing a membrane between the cis well and middle well.

The method of any of the previous embodiments, wherein the membrane between the cis well and middle well is a lipid membrane.

The method of any of the previous embodiments, further comprising depositing a protein into the membrane between the cis well and middle well, thereby forming a nanopore through the membrane.

In some embodiments, another method of manufacturing the nanopore sequencing device is disclosed. In some embodiments, the method comprises providing a first substrate comprising a dielectric layer and at least one sensing electrode on a surface of the first substrate; forming at least one nanochannel on the surface of the first substrate; depositing a sacrificial material into the at least one nanochannel; forming a first patterned layer over the substrate, wherein the first patterned layer comprises a trans well adjacent to the at least one nanochannel and at least one middle well above the sensing electrode; and removing the sacrificial material, thereby opening the at least one nanochannel.

The method of any of the previous embodiments, wherein the at least one nanochannel is formed along the surface of the first substrate without forming a through-hole in the substrate.

The method of any of the previous embodiments, wherein forming at least one nanochannel comprises etching the nanochannel into the surface of the first substrate.

The method of any of the previous embodiments, wherein forming at least one nanochannel comprises forming a patterned nanochannel structure on the surface of the first substrate.

The method of any of the previous embodiments, wherein forming a first patterned layer comprises depositing a patterning material layer over the substrate, and patterning the patterning material layer to expose the at least one sensing electrode and openings to the at least one nanochannel.

The method of any of the previous embodiments, further comprising forming an oxide or nitride layer in the at least one nanochannel, thereby reducing the width of the nanochannel.

The method of any of the previous embodiments, further comprising depositing a capping layer over the first substrate prior to forming the first patterned layer; and patterning the capping layer to expose the at least one sensing electrode and openings to the at least one nanochannel.

The method of any of the previous embodiments, wherein the trans well and the middle well are positioned side-by-side on the first substrate.

The method of any of the previous embodiments, wherein the first patterned layer further comprises a cis well next to and positioned side-by-side to the at least one middle well.

The method of any of the previous embodiments, further comprising providing a second substrate have a second patterned layer attached; and bonding the second patterned layer with the first patterned layer, thereby further define the cis well, the middle well, and the trans well between the first substrate and the second substrate.

The method of any of the previous embodiments, wherein the second substrate further comprises fluidic inlet and/or outlet holes.

The method of any of the previous embodiments, further comprising introducing a membrane between the cis well and middle well.

The method of any of the previous embodiments, wherein the membrane between the cis well and middle well is a lipid membrane.

The method of any of the previous embodiments, further comprising depositing a protein into the membrane between the cis well and middle well, thereby forming a nanopore through the membrane.

In some embodiments, a method of manufacturing the nanopore sequencing device is disclosed. In some embodiments, the method comprises providing a first substrate comprising a dielectric layer and at least one sensing electrode on a surface of the first substrate; forming a trans well in the dielectric layer; and forming at least one nanochannel on the surface of the first substrate between the trans well and the at least one sensing electrode.

The method of any of the previous embodiments, further comprising depositing a pattering material layer over the substrate; and patterning the patterning material layer to form a patterned layer comprising at least one middle well above the at least one sensing electrode, wherein the middle well is in fluid communication with the trans well through the at least one nanochannel.

The method of any of the previous embodiments, wherein the trans well is a common trans well that is in fluid communication with a plurality of middle wells through a plurality of the nanochannels.

The method of any of the previous embodiments, wherein the pattering material layer comprises a dry film photoresist.

The method of any of the previous embodiments, wherein the first patterned layer further comprises a cis well next to and positioned side-by-side to the at least one middle well.

The systems, devices, kits, and methods disclosed herein each have several aspects, no single one of which is solely responsible for their desirable attributes. Without limiting the scope of the claims, some prominent features will now be discussed briefly. Numerous other examples are also contemplated, including examples that have fewer, additional, and/or different components, steps, features, objects, benefits, and advantages. The components, aspects, and steps may also be arranged and ordered differently. After considering this discussion, and particularly after reading the section entitled “Detailed Description,” one will understand how the features of the devices and methods disclosed herein provide advantages over other known devices and methods.

It is to be understood that any features of the device and/or of the array disclosed herein may be combined together in any desirable manner and/or configuration. Further, it is to be understood that any features of the method of using the device may be combined together in any desirable manner. Moreover, it is to be understood that any combination of features of this method and/or of the device and/or of the array may be used together, and/or may be combined with any of the examples disclosed herein. Still further, it is to be understood that any feature or combination of features of any of the devices and/or of the arrays and/or of any of the methods may be combined together in any desirable manner, and/or may be combined with any of the examples disclosed herein.

It should be appreciated that all combinations of the foregoing concepts and additional concepts discussed in greater detail below are contemplated as being part of the inventive subject matter disclosed herein and may be used to achieve the benefits and advantages described herein.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A is a cross-sectional side view of an example nanopore sequencing device.

FIG. 1B illustrates another example for generating water electrolysis.

FIG. 2 shows a schematic circuit diagram of the electrical resistance provided by the nanopore sequencing device of FIG. 1A.

FIG. 3A is a cross-sectional top view of the nanopore sequencing device of FIG. 1A.

FIG. 3B is a cross-sectional top view of the nanopore sequencing device of FIG. 1A having an alternative nanochannel structure.

FIG. 4 is a cross-sectional top view of an example sequencing system utilizing the nanopore sequencing device of FIG. 1A.

FIG. 5A to FIG. 5M illustrate an example process flow of manufacturing a nanopore sequencing device.

FIG. 6 illustrates yet another example nanopore sequencing device which can generate water electrolysis.

FIG. 7A illustrates an example nanopore sequencing device with a resistive heater.

FIG. 7B is a cross-sectional top view of another example sequencing system having a pair of electrodes for generating water electrolysis.

FIG. 8 is a cross-sectional top view of another example sequencing system utilizing the nanopore sequencing device of FIG. 1A.

FIG. 9A illustrates a cross-sectional view of a portion of an alternative embodiment of a nanopore sequencing device where membranes are formed horizontally.

FIG. 9B illustrates a top view of an alternative embodiment of a nanopore sequencing device where membranes are formed horizontally.

FIG. 9C illustrates another top view of an alternative embodiment of a nanopore sequencing device where membranes are formed horizontally.

FIG. 10 is a cross-sectional view of a portion of another embodiment of a nanopore sequencing device showing the relative positions of the trans well and the middle well.

### DETAILED DESCRIPTION

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

### Definitions

All technical and scientific terms used herein have the same meaning as commonly understood to one of ordinary skill in the art to which this disclosure belongs unless clearly indicated otherwise.

As used herein, the singular forms “a”, “and”, and “the” include plural referents unless the context clearly dictates otherwise. Thus, for example, reference to “a sequence” may include a plurality of such sequences, and so forth.

The terms comprising, including, containing and various forms of these terms are synonymous with each other and are meant to be equally broad. Moreover, unless explicitly stated to the contrary, examples comprising, including, or having an element or a plurality of elements having a particular property may include additional elements, whether or not the additional elements have that property.

As used herein, the terms “fluidically connecting,” “fluid communication,” “fluidically coupled,” and the like refer to two spatial regions being connected together such that a fluid (e.g., liquid or gas) may flow between the two spatial regions. For example, a cis well/wells may be fluidically connected to a trans well/wells by way of a middle well and/or a nanochannel, such that a fluid, e.g., at least a portion of an electrolyte, may flow between the connected wells.

As used herein, the term “ionic connection” and the like refer to two spatial regions being connected together such that certain species of ions may flow between the two spatial regions.

As used herein, the term “electric connection” and the like refer to two spatial regions being connected together such that electrons, holes, ions or other charge carriers may flow between the two spatial regions.

If an electrolyte flows between two connected wells, ions and electric currents may also flow between the connected wells. In some examples, two spatial regions may be in fluid/ionic/electric communication through first and second nanoscale openings, or through one or more valves, restrictors, or other fluidic components that are to control or regulate a flow of fluid, ions or electric current through a system.

As used herein, the term “operably connected” refers to a configuration of elements, wherein an action or reaction of one element affects another element, but in a manner that preserves each element's functionality.

As used herein, the term “membrane” refers to a non-permeable or semi-permeable barrier or other sheet that separates two liquid/gel chambers (e.g., a cis well and a fluidic cavity or reservoir) which can contain the same compositions or different compositions therein. The permeability of the membrane to any given species depends upon the nature of the membrane. In some examples, the membrane may be non-permeable to ions, to electric current, and/or to fluids. For example, a lipid membrane may be impermeable to ions (i.e., does not allow any ion transport therethrough), but may be at least partially permeable to water (e.g., water diffusivity ranges from about 40 μm/s to about 100 μm/s). For another example, a synthetic/solid-state membrane, one example of which is silicon nitride, may be impermeable to ions, electric charge, and fluids (i.e., the diffusion of all of these species is zero). Any membrane may be used in accordance with the present disclosure, as long as the membrane can include a transmembrane nanoscale opening and can maintain a potential difference across the membrane. The membrane may be a monolayer or a multilayer membrane. A multilayer membrane includes two or more layers, each of which is a non-permeable or semi-permeable material.

The membrane may be formed of materials of biological or non-biological origin. A material that is of biological origin refers to material derived from or isolated from a biological environment such as an organism or cell, or a synthetically manufactured version of a biologically available structure (e.g., a biomimetic material).

An example membrane that is made from the material of biological origin includes a monolayer formed by a bolalipid. Another example membrane that is made from the material of biological origin includes a lipid bilayer. Suitable lipid bilayers include, for example, a membrane of a cell, a membrane of an organelle, a liposome, a planar lipid bilayer, and a supported lipid bilayer. A lipid bilayer can be formed, for example, from two opposing layers of phospholipids, which are arranged such that their hydrophobic tail groups face towards each other to form a hydrophobic interior, whereas the hydrophilic head groups of the lipids face outwards towards the aqueous environment on each side of the bilayer. Lipid bilayers also can be formed, for example, by a method in which a lipid monolayer is carried on an aqueous solution/air interface past either side of an aperture that is substantially perpendicular to that interface. The lipid is normally added to the surface of an aqueous electrolyte solution by first dissolving it in an organic solvent and then allowing a drop of the solvent to evaporate on the surface of the aqueous solution on either side of the aperture. Once the organic solvent has at least partially evaporated, the solution/air interfaces on either side of the aperture are physically moved up and down past the aperture until a bilayer is formed. Other suitable methods of bilayer formation include tip-dipping, painting bilayers, and patch-clamping of liposome bilayers. Any other methods for obtaining or generating lipid bilayers may also be used.

A material that is not of biological origin may also be used as the membrane. Some of these materials are solid-state materials and can form a solid-state membrane, and others of these materials can form a thin liquid film or membrane. The solid-state membrane can be a monolayer, such as a coating or film on a supporting substrate (i.e., a solid support), or a freestanding element. The solid-state membrane can also be a composite of multilayered materials in a sandwich configuration. Any material not of biological origin may be used, as long as the resulting membrane can include a transmembrane nanoscale opening and can maintain a potential difference across the membrane. The membranes may include organic materials, inorganic materials, or both. Examples of suitable solid-state materials include, for example, microelectronic materials, insulating materials (e.g., silicon nitride (Si₃N₄), aluminum oxide (Al₂O₃), hafnium oxide (HfO₂), tantalum pentoxide (Ta₂O₅), silicon oxide (SiO₂), etc.), some organic and inorganic polymers (e.g., polyamide, plastics, such as polytetrafluoroethylene (PTFE), or elastomers, such as two-component addition-cure silicone rubber), and glasses. In addition, the solid-state membrane can be made from a monolayer of graphene, which is an atomically thin sheet of carbon atoms densely packed into a two-dimensional honeycomb lattice, a multilayer of graphene, or one or more layers of graphene mixed with one or more layers of other solid-state materials. A graphene-containing solid-state membrane can include at least one graphene layer that is a graphene nanoribbon or graphene nanogap, which can be used as an electrical sensor to characterize the target polynucleotide. It is to be understood that the solid-state membrane can be made by any suitable method, for example, chemical vapor deposition (CVD). In an example, a graphene membrane can be prepared through either CVD or exfoliation from graphite. Examples of suitable thin liquid film materials that may be used include diblock copolymers or triblock copolymers, such as amphiphilic PMOXA-PDMS-PMOXA ABA triblock copolymers.

As used herein, the term “nanopore” is intended to mean a hollow structure discrete from, or defined in, and extending across the membrane. The nanopore permits ions, electric current, and/or fluids to cross from one side of the membrane to the other side of the membrane. For example, a membrane that inhibits the passage of ions or water-soluble molecules can include a nanopore structure that extends across the membrane to permit the passage (through a nanoscale opening extending through the nanopore structure) of the ions or water-soluble molecules from one side of the membrane to the other side of the membrane. The diameter of the nanoscale opening extending through the nanopore structure can vary along its length (i.e., from one side of the membrane to the other side of the membrane), but at any point is on the nanoscale (i.e., from about 1 nm to about 100 nm, or to less than 1000 nm). Examples of the nanopore include, for example, biological nanopores, solid-state nanopores, and biological and solid-state hybrid nanopores.

As used herein, the term “diameter” is intended to mean a longest straight line inscribable in a cross-section of a nanoscale opening through a centroid of the cross-section of the nanoscale opening. It is to be understood that the nanoscale opening may or may not have a circular or substantially circular cross-section. Further, the cross-section may be regularly or irregularly shaped.

As used herein, the term “biological nanopore” is intended to mean a nanopore whose structure portion is made from materials of biological origin. Biological origin refers to a material derived from or isolated from a biological environment such as an organism or cell, or a synthetically manufactured version of a biologically available structure. Biological nanopores include, for example, polypeptide nanopores and polynucleotide nanopores.

As used herein, the term “polypeptide nanopore” is intended to mean a protein/polypeptide that extends across the membrane, and permits ions, electric current, biopolymers such as DNA or peptides, or other molecules of appropriate dimension and charge, and/or fluids to flow therethrough from one side of the membrane to the other side of the membrane. A polypeptide nanopore can be a monomer, a homopolymer, or a heteropolymer. Structures of polypeptide nanopores include, for example, an α-helix bundle nanopore and a β-barrel nanopore. Example polypeptide nanopores include α-hemolysin, Mycobacterium smegmatis porin A (MspA), gramicidin A, maltoporin, OmpF, OmpC, PhoE, Tsx, F-pilus, aerolysin, etc. The protein α-hemolysin is found naturally in cell membranes, where it acts as a pore for ions or molecules to be transported in and out of cells. Mycobacterium smegmatis porin A (MspA) is a membrane porin produced by Mycobacteria, which allows hydrophilic molecules to enter the bacterium. MspA forms a tightly interconnected octamer and transmembrane beta-barrel that resembles a goblet and contains a central pore.

A polypeptide nanopore can be synthetic. A synthetic polypeptide nanopore includes a protein-like amino acid sequence that does not occur in nature. The protein-like amino acid sequence may include some of the amino acids that are known to exist but do not form the basis of proteins (i.e., non-proteinogenic amino acids). The protein-like amino acid sequence may be artificially synthesized rather than expressed in an organism and then purified/isolated.

As used herein, the term “polynucleotide nanopore” is intended to include a polynucleotide that extends across the membrane, and permits ions, electric current, and/or fluids to flow from one side of the membrane to the other side of the membrane. A polynucleotide pore can include, for example, a polynucleotide origami (e.g., nanoscale folding of DNA to create the nanopore).

Also as used herein, the term “solid-state nanopore” is intended to mean a nanopore whose structure portion is defined by a solid-state membrane and includes materials of non-biological origin (i.e., not of biological origin). A solid-state nanopore can be formed of an inorganic or organic material. Solid-state nanopores include, for example, silicon nitride nanopores, silicon dioxide nanopores, and graphene nanopores.

The nanopores disclosed herein may be hybrid nanopores. A “hybrid nanopore” refers to a nanopore including materials of both biological and non-biological origins. An example of a hybrid nanopore includes a polypeptide-solid-state hybrid nanopore and a polynucleotide-solid-state nanopore.

In some embodiments, the nanopore may comprise a solid-state material, such as silicon nitride, modified silicon nitride, silicon, silicon oxide, or graphene, or a combination thereof. In some embodiments, the nanopore is a protein that forms a tunnel upon insertion into a bilayer, membrane, thin film, or solid-state aperture. In some embodiments, the nanopore is comprised in a lipid bilayer. In some embodiments, the nanopore is comprised in an artificial membrane comprising a mycolic acid. The nanopore may be a Mycobacterium smegmatis porin (Msp) having a vestibule and a constriction zone that define the tunnel. The Msp porin may be a mutant MspA porin. In some embodiments, amino acids at positions 90, 91, and 93 of the mutant MspA porin are each substituted with asparagine. Some embodiments may comprise altering the translocation velocity or sequencing sensitivity by removing, adding, or replacing at least one amino acid of an Msp porin. A “mutant MspA porin” is a multimer complex that has at least or at most 70, 75, 80, 85, 90, 95, 98, or 99 percent or more identity, or any range derivable therein, but less than 100%, to its corresponding wild-type MspA porin and retains tunnel-forming capability. A mutant MspA porin may be recombinant protein. Optionally, a mutant MspA porin is one having a mutation in the constriction zone or the vestibule of a wild-type MspA porin. Optionally, a mutation may occur in the rim or the outside of the periplasmic loops of a wild-type MspA porin. A mutant MspA porin may be employed in any embodiment described herein.

A “vestibule” refers to the cone-shaped portion of the interior of an Msp porin whose diameter generally decreases from one end to the other along a central axis, where the narrowest portion of the vestibule is connected to the constriction zone. A vestibule may also be referred to as a “goblet.” The vestibule and the constriction zone together define the tunnel of an Msp porin. A “constriction zone” or the “readhead” refers to the narrowest portion of the tunnel of an Msp porin, in terms of diameter, that is connected to the vestibule. The length of the constriction zone may range from about 0.3 nm to about 2 nm. Optionally, the length is about, at most about, or at least about 0.3, 0.4, 0.5, 0.6, 0.7, 0.8, 0.9, 1.0, 1.1, 1.2, 1.3, 1.4, 1.5, 1.6, 1.7, 1.8, 1.9, 2, or 3 nm, or any range derivable therein. The diameter of the constriction zone may range from about 0.3 nm to about 2 nm. Optionally, the diameter is about, at most about, or at least about 0.3, 0.4, 0.5, 0.6, 0.7, 0.8, 0.9, 1.0, 1.1, 1.2, 1.3, 1.4, 1.5, 1.6, 1.7, 1.8, 1.9, 2, or 3 nm, or any range derivable therein. A “tunnel” refers to the central, empty portion of an Msp porin that is defined by the vestibule and the constriction zone, through which a gas, liquid, ion, or analyte may pass. A tunnel is an example of an opening of a nanopore.

Various conditions such as light and the liquid medium that contacts a nanopore, including its pH, buffer composition, detergent composition, and temperature, may affect the behavior of the nanopore, particularly with respect to its conductance through the tunnel as well as the movement of an analyte with respect to the tunnel, either temporarily or permanently.

In some embodiments, the disclosed system for nanopore sequencing comprises an Msp porin having a vestibule and a constriction zone that define a tunnel, wherein the tunnel is positioned between a first liquid medium and a second liquid medium, wherein at least one liquid medium comprises an analyte polynucleotide, and wherein the system is operative to detect a property of the analyte. The system may be operative to detect a property of any analyte comprising subjecting an Msp porin to an electric field such that the analyte interacts with the Msp porin. The system may be operative to detect a property of the analyte comprising subjecting the Msp porin to an electric field such that the analyte electrophoretically translocates through the tunnel of the Msp porin. In some embodiments, the system comprises an Msp porin having a vestibule and a constriction zone that define a tunnel, wherein the tunnel is positioned in a lipid bilayer between a first liquid medium and a second liquid medium, and wherein the only point of liquid communication between the first and second liquid media occurs in the tunnel. Moreover, any Msp porin described herein may be comprised in any system described herein.

The system may further comprise one or more temperature regulating devices in communication with the fluid or electrolyte. The system described herein may be operative to translocate an analyte through an Msp porin tunnel either electrophoretically or otherwise.

As used herein, the term “nanopore sequencer” refers to any of the devices disclosed herein that can be used for nanopore sequencing. In the examples disclosed herein, during nanopore sequencing, the nanopore is immersed in example(s) of the electrolyte disclosed herein and a potential difference is applied across the membrane. In an example, the potential difference is an electric potential difference or an electrochemical potential difference. An electrical potential difference can be imposed across the membrane via a voltage source that injects or administers current to at least one of the ions of the electrolyte contained in the cis well or one or more of the trans wells. An electrochemical potential difference can be established by a difference in ionic composition of the cis and trans wells in combination with an electrical potential. The different ionic composition can be, for example, different ions in each well or different concentrations of the same ions in each well.

The application of the potential difference across a nanopore may force the translocation of a nucleic acid through the nanopore. One or more signals are generated that correspond to the translocation of the nucleotide through the nanopore. Accordingly, as a target polynucleotide, or as a mononucleotide or a probe derived from the target polynucleotide or mononucleotide, transits through the nanopore, the current across the membrane changes due to base-dependent (or probe dependent) blockage of the constriction, for example. The signal from that change in current can be measured using any of a variety of methods. Each signal is unique to the species of nucleotide(s) (or probe) in the nanopore, such that the resultant signal can be used to determine a characteristic of the polynucleotide. For example, the identity of one or more species of nucleotide(s) (or probe) that produces a characteristic signal can be determined.

As used herein, a “reporter” is composed of one or more reporter elements. Reporters include what are known as “tags” and “labels.” Reporters serve to parse the genetic information of the target nucleic acid. “Encode” or “parse” are verbs referring to transferring from one format to another, and refers to transferring the genetic information of target template base sequence into an arrangement of reporters.

As used herein, a “peptide” refers to two or more amino acids joined together by an amide bond (that is, a “peptide bond”). Peptides comprise up to or include 50 amino acids. Peptides may be linear or cyclic. Peptides may be α, β, γ, δ, or higher, or mixed. Peptides may comprise any mixture of amino acids as defined herein, such as comprising any combination of D, L, α, β, γ, δ, or higher amino acids.

As used herein, a “protein” refers to an amino acid sequence having 51 or more amino acids.

As used herein, a “polymerase” is an enzyme generally used for joining 3′-OH 5′-triphosphate nucleotides, oligomers, and their analogs. Polymerases include, but are not limited to, DNA-dependent DNA polymerases, DNA-dependent RNA polymerases, RNA-dependent DNA polymerases, RNA-dependent RNA polymerases, T7 DNA polymerase, T3 DNA polymerase, T4 DNA polymerase, T7 RNA polymerase, T3 RNA polymerase, SP6 RNA polymerase, DNA polymerase I, Klenow fragment, Thermophilus aquaticus DNA polymerase, Tth DNA polymerase, VentR® DNA polymerase (New England Biolabs), Deep VentR® DNA polymerase (New England Biolabs), Bst DNA Polymerase Large Fragment, Stoeffel Fragment, 90N DNA Polymerase, 90N DNA polymerase, Pfu DNA Polymerase, TfI DNA Polymerase, Tth DNA Polymerase, RepliPHI Phi29 Polymerase, TIi DNA polymerase, eukaryotic DNA polymerase beta, telomerase, Therminator™ polymerase (New England Biolabs), KOD HiFi™ DNA polymerase (Novagen), KODI DNA polymerase, Q-beta replicase, terminal transferase, AMV reverse transcriptase, M-MLV reverse transcriptase, Phi6 reverse transcriptase, HIV-1 reverse transcriptase, novel polymerases discovered by bioprospecting, and polymerases cited in US 2007/0048748, U.S. Pat. Nos. 6,329,178, 6,602,695, and 6,395,524 (incorporated by reference). These polymerases include wild-type, mutant isoforms, and genetically engineered variants.

As used herein, a “nucleotide” includes a nitrogen containing heterocyclic base, a sugar, and one or more phosphate groups. Nucleotides are monomeric units of a nucleic acid sequence. Examples of nucleotides include, for example, ribonucleotides or deoxyribonucleotides. In ribonucleotides (RNA), the sugar is a ribose, and in deoxyribonucleotides (DNA), the sugar is a deoxyribose, i.e., a sugar lacking a hydroxyl group that is present at the 2′ position in ribose. The nitrogen containing heterocyclic base can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. The phosphate groups may be in the mono-, di-, or tri-phosphate form. These nucleotides are natural nucleotides, but it is to be further understood that non-natural nucleotides, modified nucleotides or analogs of the aforementioned nucleotides can also be used.

As used herein, “nucleobase” is a heterocyclic base such as adenine, guanine, cytosine, thymine, uracil, inosine, xanthine, hypoxanthine, or a heterocyclic derivative, analog, or tautomer thereof. A nucleobase can be naturally occurring or synthetic. Non-limiting examples of nucleobases are adenine, guanine, thymine, cytosine, uracil, xanthine, hypoxanthine, 8-azapurine, purines substituted at the 8 position with methyl or bromine, 9-oxo-N6-methyladenine, 2-aminoadenine, 7-deazaxanthine, 7-deazaguanine, 7-deaza-adenine, N4-cthanocytosine, 2,6-diaminopurine, N6-ethano-2,6-diaminopurine, 5-methylcytosine, 5-(C3-C6)-alkynylcytosine, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6-dihydrothymine, 5,6-dihydrouracil, 4-methyl-indole, ethenoadenine and the non-naturally occurring nucleobases described in U.S. Pat. Nos. 5,432,272 and 6,150,510 and PCT applications WO 92/002258, WO 93/10820, WO 94/22892, and WO 94/24144, and Fasman (“Practical Handbook of Biochemistry and Molecular Biology”, pp. 385-394, 1989, CRC Press, Boca Raton, LO), all herein incorporated by reference in their entireties.

The term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in manner similar to naturally occurring nucleotides, such as peptide nucleic acids (PNAs) and phosphorothioate DNA. Unless otherwise indicated, a particular nucleic acid sequence includes the complementary sequence thereof. Nucleotides include, but are not limited to, ATP, dATP, CTP, dCTP, GTP, dGTP, UTP, TTP, dUTP, 5-methyl-CTP, 5-methyl-dCTP, ITP, dITP, 2-amino-adenosine-TP, 2-amino-deoxyadenosine-TP, 2-thiothymidine triphosphate, pyrrolo-pyrimidine triphosphate, and 2-thiocytidine, as well as the alphathiotriphosphates for all of the above, and 2′-O-methyl-ribonucleotide triphosphates for all the above bases. Modified bases include, but are not limited to, 5-Br-UTP, 5-Br-dUTP, 5-F-UTP, 5-F-dUTP, 5-propynyl dCTP, and 5-propynyl-dUTP.

For example, a template polynucleotide chain may be any sample that is to be sequenced, and may be composed of DNA, RNA, or analogs thereof (e.g., peptide nucleic acids). The source of the template (or target) polynucleotide chain can be genomic DNA, messenger RNA, or other nucleic acids from native sources. In some cases, the template polynucleotide chain that is derived from such sources can be amplified prior to use. Any of a variety of known amplification techniques can be used including, but not limited to, polymerase chain reaction (PCR), rolling circle amplification (RCA), multiple displacement amplification (MDA), or random primer amplification (RPA). It is to be understood that amplification of the template polynucleotide chain prior to use is optional. As such, the template polynucleotide chain will not be amplified prior to use in some examples. Template/target polynucleotide chains can optionally be derived from synthetic libraries. Synthetic nucleic acids can have native DNA or RNA compositions or can be analogs thereof.

Biological samples from which the template polynucleotide chain can be derived include, for example, those from a mammal, such as a rodent, mouse, rat, rabbit, guinea pig, ungulate, horse, sheep, pig, goat, cow, cat, dog, primate, human or non-human primate; a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a Dictyostelium discoideum; a fungi such as Pneumocystis carinii Takifugu rubripes, yeast, Saccharomyces cerevisiae or Schizosaccharomyces pombe; or a Plasmodium falciparum. Template polynucleotide chains 48 can also be derived from prokaryotes such as a bacterium, Escherichia coli, staphylococci or Mycoplasma pneumoniae; an archaea; a virus such as Hepatitis C virus, Ebola virus or human immunodeficiency virus; or a viroid. Template polynucleotide chains can be derived from a homogeneous culture or population of the above organisms or alternatively from a collection of several different organisms, for example, in a community or ecosystem.

Moreover, template polynucleotide chains may not be derived from natural sources, but rather can be synthesized using known techniques. For example, gene expression probes or genotyping probes can be synthesized and used in the examples set forth herein.

In some examples, template polynucleotide chains can be obtained as fragments of one or more larger nucleic acids. Fragmentation can be carried out using any of a variety of techniques known in the art including, for example, nebulization, sonication, chemical cleavage, enzymatic cleavage, or physical shearing. Fragmentation may also result from use of a particular amplification technique that produces amplicons by copying only a portion of a larger nucleic acid chain. For example, PCR amplification produces fragments having a size defined by the length of the nucleotide sequence on the original template that is between the locations where flanking primers hybridize during amplification. The length of the template polynucleotide chain may be in terms of the number of nucleotides or in terms of a metric length (e.g., nanometers).

A population of template/target polynucleotide chains, or amplicons thereof, can have an average strand length that is desired or appropriate for a particular sequencing device. For example, the average strand length can be less than about 100,000 nucleotides, about 50,000 nucleotides, about 10,000 nucleotides, about 5,000 nucleotides, about 1,000 nucleotides, about 500 nucleotides, about 100 nucleotides, or about 50 nucleotides. Alternatively or additionally, the average strand length can be greater than about 10 nucleotides, about 50 nucleotides, about 100 nucleotides, about 500 nucleotides, about 1,000 nucleotides, about 5,000 nucleotides, about 10,000 nucleotides, about 50,000 nucleotides, or about 100,000 nucleotides. Alternatively or additionally, the average strand length can be greater than about 10 kilo nucleotides, about 50 kilo nucleotides, about 100 kilo nucleotides, about 500 kilo nucleotides, about 1,000 kilo nucleotides, about 5,000 kilo nucleotides, about 10,000 kilo nucleotides, about 50,000 kilo nucleotides, or about 100,000 kilo nucleotides. Alternatively or additionally, the average strand length can be greater than about 10 mega nucleotides, about 50 mega nucleotides, about 100 mega nucleotides, about 500 mega nucleotides, about 1,000 mega nucleotides, about 5,000 mega nucleotides, about 10,000 mega nucleotides, about 50,000 mega nucleotides, or about 100,000 mega nucleotides. The average strand length for a population of target polynucleotide chains, or amplicons thereof, can be in a range between a maximum and minimum value set forth above.

In some cases, a population of template/target polynucleotide chains can be produced under conditions or otherwise configured to have a maximum length for its members. For example, the maximum length for the members can be less than about 100,000 nucleotides, about 50,000 nucleotides, about 10,000 nucleotides, about 5,000 nucleotides, about 1,000 nucleotides, about 500 nucleotides, about 100 nucleotides or about 50 nucleotides. For example, the maximum length for the members can be less than about 100,000 kilo nucleotides, about 50,000 kilo nucleotides, about 10,000 kilo nucleotides, about 5,000 kilo nucleotides, about 1,000 kilo nucleotides, about 500 kilo nucleotides, about 100 kilo nucleotides or about 50 kilo nucleotides. For example, the maximum length for the members can be less than about 100,000 mega nucleotides, about 50,000 mega nucleotides, about 10,000 mega nucleotides, about 5,000 mega nucleotides, about 1,000 mega nucleotides, about 500 mega nucleotides, about 100 mega nucleotides or about 50 mega nucleotides. Alternatively or additionally, a population of template polynucleotide chains, or amplicons thereof, can be produced under conditions or otherwise configured to have a minimum length for its members. For example, the minimum length for the members can be more than about 10 nucleotides, about 50 nucleotides, about 100 nucleotides, about 500 nucleotides, about 1,000 nucleotides, about 5,000 nucleotides, about 10,000 nucleotides, about 50,000 nucleotides, or about 100,000 nucleotides. For example, the minimum length for the members can be more than about 10 kilo nucleotides, about 50 kilo nucleotides, about 100 kilo nucleotides, about 500 kilo nucleotides, about 1,000 kilo nucleotides, about 5,000 kilo nucleotides, about 10,000 kilo nucleotides, about 50,000 kilo nucleotides, or about 100,000 kilo nucleotides. For example, the minimum length for the members can be more than about 10 mega nucleotides, about 50 mega nucleotides, about 100 mega nucleotides, about 500 mega nucleotides, about 1,000 mega nucleotides, about 5,000 mega nucleotides, about 10,000 mega nucleotides, about 50,000 mega nucleotides, or about 100,000 mega nucleotides. The maximum and minimum strand length for template polynucleotide chains in a population can be in a range between a maximum and minimum value set forth above.

As used herein, the term “signal” is intended to mean an indicator that represents information. Signals include, for example, an electrical signal and an optical signal. The term “electrical signal” refers to an indicator of an electrical quality that represents information. The indicator can be, for example, current, voltage, tunneling, resistance, potential, voltage, conductance, or a transverse electrical effect (and any time-derivatives or transients of theses). An “electronic current” or “electric current” refers to a flow of electric charge. In an example, an electrical signal may be an electric current passing through a nanopore, and the electric current may flow when an electric potential difference is applied across the nanopore.

The term “substrate” refers to a rigid, solid support that is insoluble in aqueous liquid and is incapable of passing a liquid absent an aperture, port, or other like liquid conduit. In the examples disclosed herein, the substrate may have wells or chambers defined therein. Examples of suitable substrates include glass and modified or functionalized glass, plastics (including acrylics, polystyrene and copolymers of styrene and other materials, polypropylene, polyethylene, polybutylene, polyurethanes, polytetrafluoroethylene (PTFE) (such as TEFLON® from Chemours), cyclic olefins/cyclo-olefin polymers (COP) (such as ZEONOR® from Zeon), polyimides, etc.), nylon, ceramics, silica or silica-based materials, silicon and modified silicon, carbon, metals, inorganic glasses, and optical fiber bundles.

As used herein, the term “interstitial region” refers to an area in a substrate/solid support or a membrane, or an area on a surface that separates other areas, regions, features associated with the support or membrane or surface. For example, an interstitial region of a membrane can separate one nanopore of an array from another nanopore of the array. For another example, an interstitial region of a substrate can separate one trans/cis well from another trans/cis well. The two areas that are separated from each other can be discrete, i.e., lacking physical contact with each other. In many examples, the interstitial region is continuous whereas the areas are discrete, for example, as is the case for a plurality of nanopores defined in an otherwise continuous membrane, or for a plurality of wells defined in an otherwise continuous substrate/support. The separation provided by an interstitial region can be partial or full separation. Interstitial regions may have a surface material that differs from the surface material of the features defined in the surface. For example, the surface material at the interstitial regions may be a lipid material, and a nanopore formed in the lipid material can have an amount or concentration of polypeptide that exceeds the amount or concentration present at the interstitial regions. In some examples, the polypeptide may not be present at the interstitial regions.

The terms top, bottom, lower, upper, on, etc. are used herein to describe the device/nanopore sequencer and/or the various components of the device. It is to be understood that these directional terms are not meant to imply a specific orientation, but are used to designate relative orientation between components. The use of directional terms should not be interpreted to limit the examples disclosed herein to any specific orientation(s). As used herein, the terms “upper”, “lower”, “vertical”, “horizontal” and the like are meant to indicate relative orientation.

As used herein, “cis” refers to the side of a nanopore opening through which an analyte or modified analyte enters the opening or across the face of which the analyte or modified analyte moves.

As used herein, “trans” refers to the side of a nanopore opening through which an analyte or modified analyte (or fragments thereof) exits the opening or across the face of which the analyte or modified analyte does not move.

As used herein, by “translocation,” it is meant that an analyte (e.g., DNA) enters one side of an opening of a nanopore and move to and out of the other side of the opening. It is contemplated that any embodiment herein comprising translocation may refer to electrophoretic translocation or non-electrophoretic translocation, unless specifically noted. An electric field may move an analyte (e.g., a polynucleotide) or modified analyte. By “interacts,” it is meant that the analyte (e.g., DNA) or modified analyte moves into and, optionally, through the opening, where “through the opening” (or “translocates”) means to enter one side of the opening and move to and out of the other side of the opening. Optionally, methods that do not employ electrophoretic translocation are contemplated. In some embodiments, physical pressure causes a modified analyte to interact with, enter, or translocate (after alteration) through the opening. In some embodiments, a magnetic bead is attached to an analyte or modified analyte on the trans side, and magnetic force causes the modified analyte to interact with, enter, or translocate (after alteration) through the opening. Other methods for translocation include but not limited to gravity, osmotic forces, temperature, and other physical forces such as centripetal force.

As used herein, the terms “well”, “cavity”, “reservoir” and “chamber” are used synonymously, and refer to a discrete feature defined in the device that can contain a fluid (e.g., liquid, gel, gas). A cis well is a chamber that contains or is partially defined by a cis electrode, and is also fluidically connected to a middle well where measurements occur (for example, by a FET, or by a metal electrode connected to an amplifier, a data acquisition device, or other signal conditioning elements such as analog filters, buffers, gain amplifiers, ADCs, etc.). The middle well in turn is fluidically connected to a trans well/chamber, in some examples. Examples of an array of the present device may have one cis well, for example one global cis chamber/reservoir, or multiple cis wells. The trans well is a single chamber that contains or is partially defined by its own trans electrode, and is also fluidically connected to a cis well. In examples including multiple trans wells, each trans well is electrically isolated from each other trans well. Further, it is to be understood that the cross-section of a well taken parallel to a surface of a substrate at least partially defining the well can be curved, square, polygonal, hyperbolic, conical, angular, etc. As used herein, “field-effect transistors” or “FETs” typically include doped source/drain regions that are formed of a semiconductor material, e.g., silicon, germanium, gallium arsenide, silicon carbide, etc., and are separated by a channel region. A n-FET is a FET having an n-channel in which the current carriers are electrons. A p-FET is a FET having a p-channel in which the current carriers are holes. Source/drain regions of a n-FET device may include a different material than source/drain regions of a p-FET device. In some examples, the source/drain regions or the channel may not be doped. Doped regions may be formed by adding dopant atoms to an intrinsic semiconductor. This changes the electron and hole carrier concentrations of the intrinsic semiconductor at thermal equilibrium. A doped region may be p-type or n-type. As used herein, “p-type” refers to the addition of impurities to an intrinsic semiconductor that creates a deficiency of valence electrons. For silicon, example p-type dopants, i.e., impurities, include but are not limited to boron, aluminum, gallium, and indium. As used herein, “n-type” refers to the addition of impurities that contribute free electrons to an intrinsic semiconductor. For silicon, example n-type dopants, i.e., impurities, include but are not limited to, antimony, arsenic, and phosphorus. The dopant(s) may be introduced by ion implantation or plasma doping.

For example, in an integrated circuit having a plurality of metal oxide semiconductor field effect transistors (MOSFETs), each MOSFET has a source and a drain that are formed in an active region of a semiconductor layer by implanting n-type or p-type impurities in the layer of semiconductor material. Disposed between the source and the drain is a channel (or body) region. Disposed above the body region is a gate electrode. The gate electrode and the body are spaced apart by a gate dielectric (gate oxide) layer. The channel region connects the source and the drain, and electrical current flows through the channel region from the source to the drain. The electrical current flow is induced in the channel region by a voltage applied at the gate electrode.

In some embodiments, the channel of a FET sensor located between the source and drain may be covered by a relatively thin layer of a gate oxide, for example a thermally grown silicon dioxide layer. Alternatively, a thin layer of an insulator may be formed of high-K dielectrics, such as HfO₂, Al₂O₃, silicon nitroxides, Si₃N₄, TiO₂, Ta₂O₅, Y₂O₃, La₂O₃, ZrO₂, ZrSiO₄, barium strontium titanate, lead zirconate titanate, ZrSiₓO_{y}, or ZrAlₓO_{y}. The layer of gate oxide may be about 10 nm in thickness, or in other examples, less than about 9, about 8, about 7, about 6, about 5, about 4, about 3, about 2, or about 1 nm in thickness.

Non-planar transistor device architectures, such as nanosheet (or nanowire) transistors, can provide increased device density and increased performance over planar transistors. A “gate-all-around” transistor is a transistor in which the gate is structured to wrap around the channel. A “nanosheet transistor” refers to a type of FET that may include a plurality of stacked nanosheets extending between a pair of source/drain regions, forming a channel. Nanosheet transistors, in contrast to conventional planar FETs, may include a gate stack that wraps around the full perimeter of multiple nanosheet channel regions. Nanosheet transistor configurations enable fuller depletion in the nanosheet channel regions and reduce short-channel effects. “Nanowire transistors” may be similar to nanosheet transistors, except the channel may include nanowires instead of nanosheets. The gate-all-around structure in nanosheet or nanowire transistors can provide very small devices with better switching control, lower leakage current, faster operations, and lower output resistance.

A way of increasing channel conductivity and decreasing FET size is to form the channel as a nanostructure. For example, a gate-all-around (GAA) nanosheet FET is an architecture for providing a relatively small FET footprint by forming the channel region as a series of nanosheets. In a GAA configuration, a nanosheet-based FET includes a source region, a drain region and stacked nanosheet channels between the source and drain regions. A gate surrounds the stacked nanosheet channels and regulates electron flow through the nanosheet channels between the source and drain regions. GAA nanosheet FETs may be fabricated by forming alternating layers of channel nanosheets and sacrificial nanosheets. The sacrificial nanosheets are released from the channel nanosheets before the FET device is finalized. For n-type FETs, the channel nanosheets are typically silicon (Si) and the sacrificial nanosheets are typically silicon germanium (SiGe). For p-type FETs, the channel nanosheets are typically SiGe and the sacrificial nanosheets are typically Si. In some implementations, the channel nanosheet of a p-FET can be SiGe or Si, and the sacrificial nanosheets can be Si or SiGe. Forming the GAA nanosheets from alternating layers of channel nanosheets formed from a first type of semiconductor material (e.g., Si for n-type FETs, and SiGe for p-type FETs) and sacrificial nanosheets formed from a second type of semiconductor material (e.g., SiGe for n-type FETs, and Si for p-type FETs) provides superior channel electrostatics control, which is beneficial for continuously scaling gate lengths down to seven nanometer CMOS technology and below. The use of multiple layered SiGe/Si sacrificial/channel nanosheets (or Si/SiGe sacrificial/channel nanosheets) to form the channel regions in GAA FET semiconductor devices provides desirable device characteristics, including the introduction of strain at the interface between SiGe and Si.

In some examples, a “nanowire” is characterized by a critical dimension of less than about 30 nm, while a “nanosheet” is characterized by a critical dimension of about 30 nm or greater. In exemplary devices, the critical dimension is measured along the gate. In that direction, if the width of the channel is small, the channel cross-section is like a “wire” whereas if the width of the channel is large, the channel cross-section is like a “sheet.”

In some examples, the smallest dimension of the nanosheet or nanowire is between about 1-10, about 1-50, about 1-100, about 1-500, or about 1-1000 nm. In some examples, the smallest dimension of the nanosheet or nanowire is between about 1-5, about 3-10, about 5-15, about 10-20, about 15-30, about 20-40, about 30-50, about 40-75, about 50-100, about 75-150, about 100-200, about 150-300, about 200-400, about 300-500, about 400-750, or about 500-1000 nm. In some examples, the smallest dimension of the nanosheet is at least about 3, about 5, about 7, about 10, about 15, about 20, about 50, about 100, about 150, about 200, about 250, about 300, about 350, about 400, about 450, about 500, about 600, about 700, about 800, about 900, about 1000, about 2000, about 2500, about 3000, about 4000, or about 5000 times smaller than the other two dimensions of the nanosheet. In some examples, the smallest dimension of the nanosheet is between about 2-5, about 3-7, about 5-10, about 7-15, about 10-20, about 15-50, about 20-100, about 50-150, about 100-200, about 150-250, about 200-300, about 250-350, about 300-400, about 350-450, about 400-500, about 450-600, 5 about 00-700, about 600-800, about 700-900, about 800-1000, about 900-2000, about 1000-2500, about 2000-3000, about 2500-4000, or about 3000-5000 times smaller than the other two dimensions of the nanosheet. In some examples, the smallest dimension of the nanosheet is at most about 3, about 5, about 7, about 10, about 15, about 20, about 50, about 100, about 150, about 200, about 250, about 300, about 350, about 400, about 450, about 500, about 600, about 700, about 800, about 900, about 1000, about 2000, about 2500, about 3000, about 4000, or about 5000 times smaller than the other two dimensions of the nanosheet. In some examples, the biggest dimension of the nanowire is at least about 3, about 5, about 7, about 10, about 15, about 20, about 50, about 100, about 150, about 200, about 250, about 300, about 350, about 400, about 450, about 500, about 600, about 700, about 800, about 900, about 1000, about 2000, about 2500, about 3000, about 4000, or about 5000 times bigger than the other two dimensions of the nanowire. In some examples, the biggest dimension of the nanowire is between about 2-5, about 3-7, about 5-10, about 7-15, about 10-20, about 15-50, about 20-100, about 50-150, about 100-200, about 150-250, about 200-300, about 250-350, about 300-400, about 350-450, about 400-500, about 450-600, about 500-700, about 600-800, about 700-900, about 800-1000, about 900-2000, about 1000-2500, about 2000-3000, about 2500-4000, or about 3000-5000 times bigger than the other two dimensions of the nanowire. In some examples, the biggest dimension of the nanowire is at most about 3, about 5, about 7, about 10, about 15, about 20, about 50, about 100, about 150, about 200, about 250, about 300, about 350, about 400, about 450, about 500, about 600, about 700, about 800, about 900, about 1000, about 2000, about 2500, about 3000, about 4000, or about 5000 times bigger than the other two dimensions of the nanowire.

The aspects and examples set forth herein and recited in the claims can be understood in view of the above definitions.

### Overview

In some aspects, the disclosure provides a process integration scheme for achieving a horizontal architecture of a nanopore sequencing device, a nanopore sequencing device having a horizontal architecture, and a method of using such a device.

Disclosed herein is a nanopore sequencing device includes a middle well associated with a sensing electrode, a cis well associated with a cis electrode, and a trans well associated with a trans electrode. In some embodiments, the middle well is positioned between the cis well and the trans well, and the cis well, middle well, and the trans well are oriented side-by-side horizontally. In some embodiments, the cis well is oriented vertically with respect to the middle and/or trans well. In some embodiments, the trans well is oriented vertically with respect to the middle well. In some embodiments, the device may comprise one or more common cis well and one or more common trans well that are shared by all sequencing unit cells. For example, a common trans wall and a common cis well may be in fluid communication with a plurality of middle wells. In some embodiments, the cis and trans wells may be considerably larger than each of the middle wells to avoid ion depletion, while each middle well may contain its own, individually addressable sensing electrode.

The nanopore sequencing device further includes a first nanoscale opening, e.g., a nanoscale opening arranged in a nanopore, disposed between the cis well and the middle well, and a second nanoscale opening, e.g., a horizontal nanochannel, formed on the surface of the substrate between the trans well and the middle well. In some embodiments, the nanochannel is fabricated horizontally on the surface of the substrate. For example, in some embodiments, the nanochannel is formed by etching a semiconductor wafer. In some embodiments, the nanochannel is formed by patterned layers over a semiconductor wafer. In some embodiments, the nanochannel does not comprise a through-hole in the substrate. The middle well of the nanopore sequencing device fluidically connects the cis well to the trans well.

When one or more nucleotides of a target DNA are near or at the first nanoscale opening, e.g., near or at the nanopore, the electrical resistance of the first nanoscale opening may vary in response to the identity of the one or more nucleotides. The second nanoscale opening, e.g., the nanochannel, may have a fixed, or substantially fixed electrical resistance. In some embodiments, the length of the nanochannel is chosen for its specific electrical resistance. In some embodiments, the electrical resistance of the first nanochannel is altered by changing the length of the nanochannel. In some embodiments, such a device may further include electronics to actively control the second nanoscale opening, e.g., to control the nanochannel. For example, pressure pulse generators, air/gas bubble generators/annihilators, stimuli-responsive polymers or gels may be used to control liquid/ionic/electric flow in the second nanoscale opening. In some embodiments, the electronics or actuators may be formed under (or around) the second nanoscale opening, e.g., formed under (or around) the nanochannel.

In some embodiments, the device may further include one or more additional middle wells, each additional middle well associated with a respective additional sensing electrode, where a respective additional first nanoscale opening is disposed between the cis well and each additional middle well, where a respective additional second nanoscale opening is disposed between the trans well and each additional middle well, and where the one or more additional middle wells fluidically connect the cis well to the trans well. In some embodiments, at least some of the additional first nanoscale openings may be arranged in nanopores. In some embodiments, at least some of the additional second nanoscale openings may be arranged in nanochannels. In some embodiments, an array of middle wells is formed on a substrate, the middle wells are in fluid communication with one or more common trans well or trans channel, and also in fluid communication with one or more common cis well or cis channel.

In some embodiments, to use such a device for sequencing a biopolymer, a method may include introducing an electrolyte into the cis well, the trans well, and at least one of the middle wells. The method may further include applying a voltage between the cis electrode and the trans electrode to control the motion of the biopolymer. The method may further include measuring, from the respective sensing electrode, an electric potential of the electrolyte in the middle well, where an electrical resistance of the respective first nanoscale opening, e.g., nanopore, varies in response to an identity of one or more monomers in the biopolymer, the one or more monomers being near or at the respective first nanoscale opening.

In some embodiments, a method of manufacturing such a device may include forming a bottom wafer comprising at least one of the second nanoscale openings, e.g., nanochannels, at least one of the respective sensing electrodes, and a first patterned layer. In some embodiments, the nanochannels are horizontal nanochannels. In some embodiments, the nanochannels are fabricated into the substrate. For example, in some embodiments, the nanochannels are etched into a semiconductor wafer. In some embodiments, the nanochannels are formed by patterned layers over a semiconductor wafer. The method may further include forming a top wafer comprising a second patterned layer. The method may further include aligning the first patterned layer with the second patterned layer. The method may further include bonding the first patterned layer with the second patterned layer at a plurality of locations via an adhesive, such that the cis well, the trans well, and at least one of the respective middle wells are formed between the bottom wafer and the top wafer.

In certain embodiments, a nanopore sequencing device having a horizontal structure includes one or a combinations of the following aspects:

(i) Ease of or more precise manufacturing process eliminating the need to (a) etch high aspect ratio through-silicon vias/cavities into the Si substrate, (b) perform backside wafer processing that may compromise the wafer front-side, (c) use expensive 193 nm lithography masks, and (d) perform wafer-to-wafer bonding. Fabricating horizontal nanochannels of specific fluidic/electric resistance reduces the number of undesirable steps (such as complex multiple etch steps, deposition of sacrificial etch stop layers, re-oxidation and wafer backside processing steps) compared to vertical through-Si nanochannels.

(ii) In terms of manufacturability and reproducibility, the uniformity of horizontal nanochannel width (or diameter), both along a single nanochannel and across nanochannels on a wafer, can be better controlled compared to a vertical implementation of nanochannels. Moreover, non-destructive metrology can be employed to assess uniformity of critical dimensions across a nanochannel and wafer.

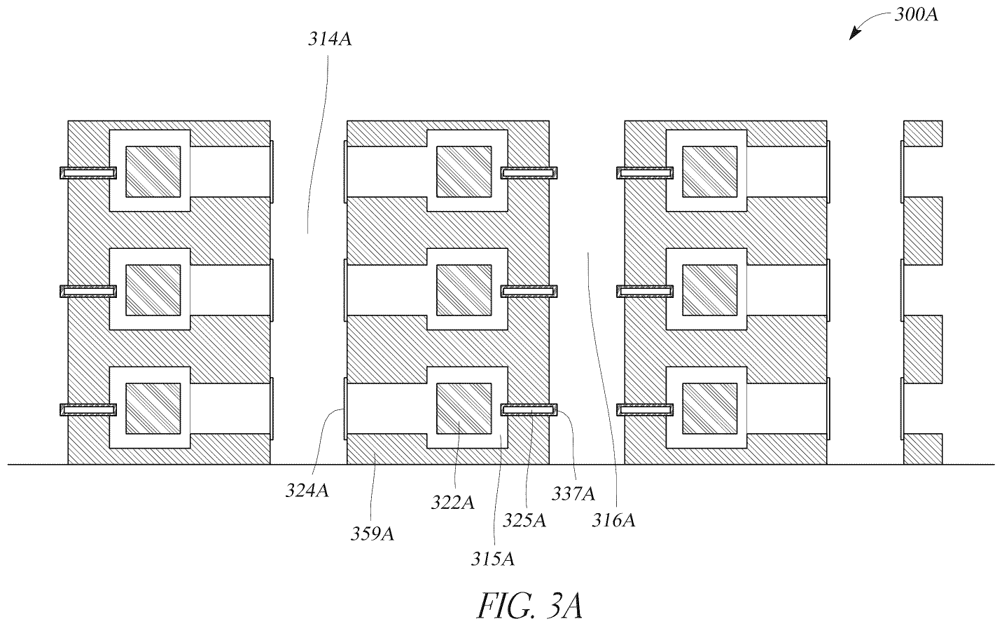

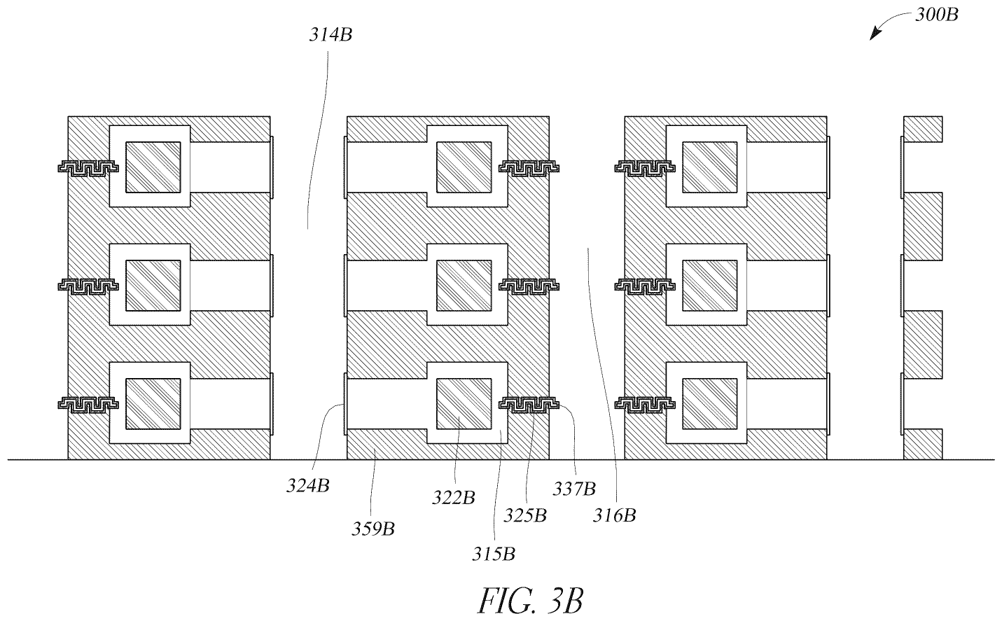

(iii) Ability to increase nanochannel resistance by increasing overall nanochannel length, e.g., using a meandering/tortuous/serpentine layout as shown in FIG. 3B instead of a linear layout. Variations of the nanochannel resistance can be implemented easily on the same substrate by changing the nanochannel length. Unlike the length of vertical nanochannels in a vertical device implementation, the length of horizontal nanochannels is not limited by the substrate thickness and multiple lengths/resistances can be achieved on one wafer thus allowing faster learning cycles and process optimization. A wider nanochannel width (or diameter) is beneficial for reducing the pressure inside the middle well, but this wider nanochannel width may need to be accompanied by an increased nanochannel length to achieve the required nanochannel resistance, which poses difficulties for a vertical device implementation relying on through-Si etch.

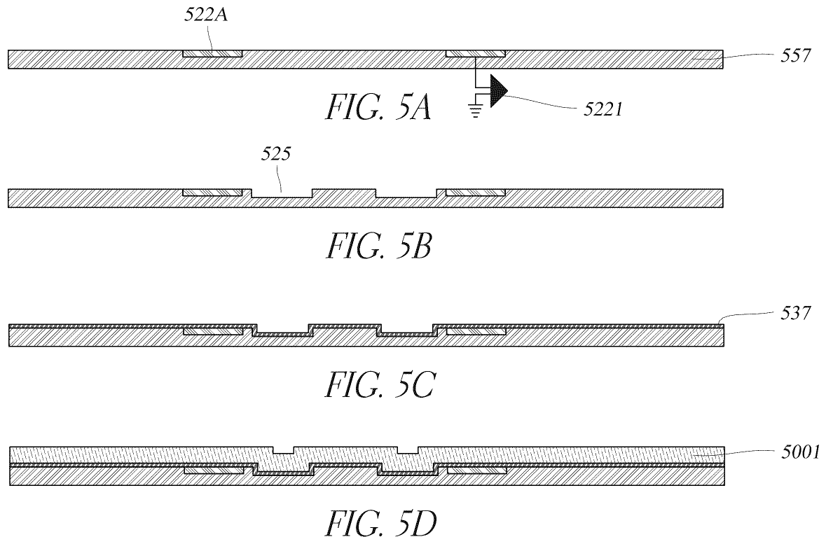

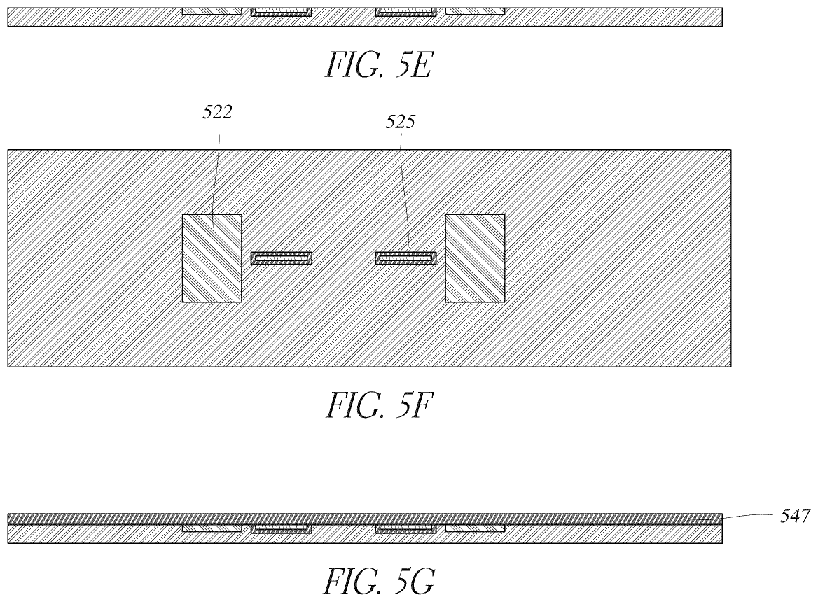

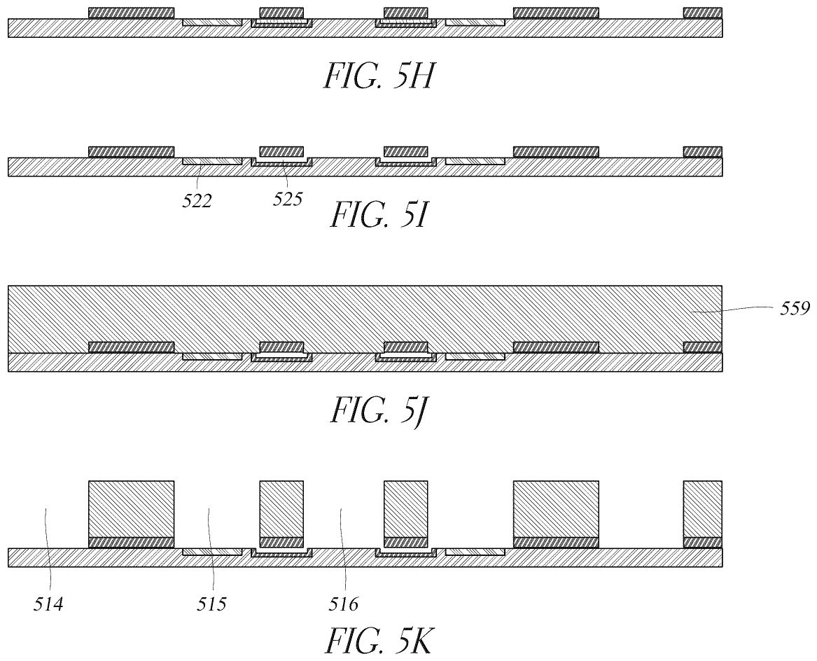

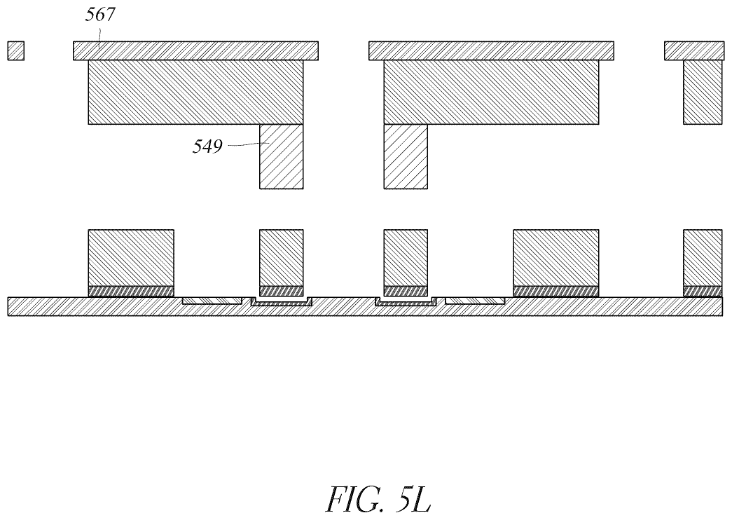

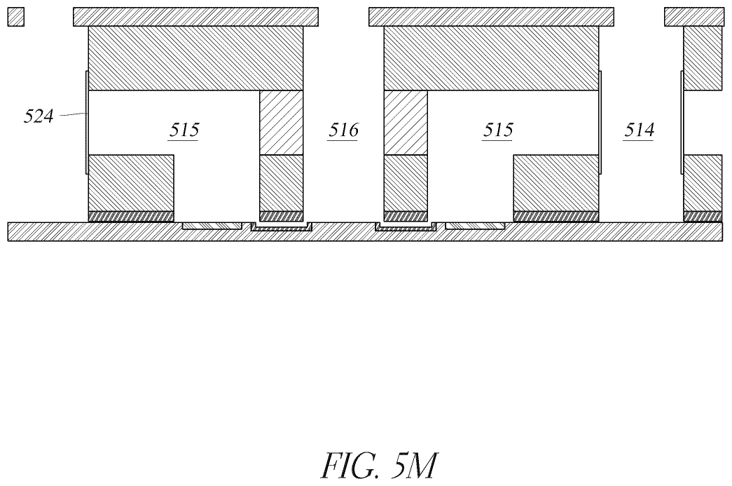

(iv) The ability to achieve longer nanochannels relaxes the nanochannel diameter and eliminates the need for subsequent deposition of thick (e.g., >500 nm) layers to reduce the nanochannel width. For example, one could start with a nanochannel width of 350 nm defined using inexpensive i-line lithography or nanoimprint lithography and deposit a 135 nm oxide or nitride layer to achieve a final nanochannel width of 80 nm, as shown in FIG. 5F′.

(v) Ability to integrate active electronics underneath the nanochannel to control resistance and/or act as an electronic switch/valve. Implementation of horizontal nanochannels also allows integration of active circuitry/actuators underneath or in the vicinity of each nanochannel to control/regulate the nanochannel resistance or other behaviors. For example, an integrated heating element (e.g., resistor) could be used to generate nanobubbles (water vapor) that block a nanochannel and thus switch off the current/ionic/fluidic flow through that respective nanochannel and sequencing unit cell. This helps avoid current/ionic/fluidic flow through sequencing unit cells which are identified as corrupted or non-functional. For another example, a piezoelectric element (e.g., ultrasonic actuator) could be used to eliminate bubbles or other unwanted debris (e.g., clogged DNA template) in the nanochannel. Other control modalities employed in digital microfluidics (such as electrowetting) or valve elements involving stimuli-responsive polymers or hydrogels may also be integrated into the nanochannel of the disclosed device.

(vi) Improved device robustness by integration of cis and trans fluidic wells onto the chip thus eliminating the need for complex setup fixtures or hardware integration.

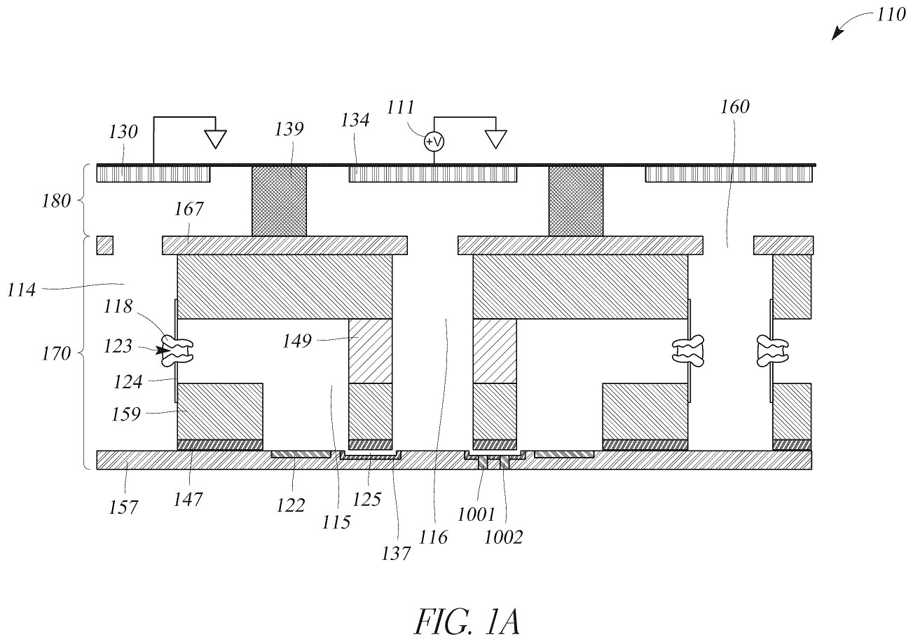

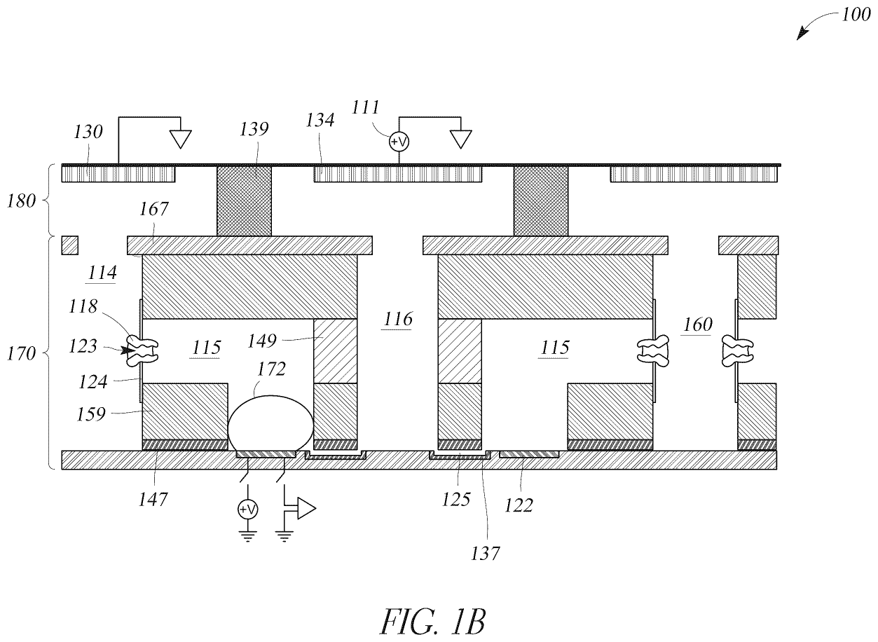

(vii) The cis and trans wells can be integrated directly onto the chip by means of wafer-to-wafer bonding, thus creating a fully contained flowcell that, in some embodiments, only requires inlet/outlet ports for fluidic and electrical connections to an external fixture/cartridge (see FIG. 1A for an example). External fluidic reservoirs and electrodes can be made large enough to maintain constant ion concentrations inside the flowcell without risk of ion depletion.

### Example Nanopore Sequencing Devices

FIG. 1A is a cross-sectional side view of an example nanopore sequencing device 110 having a horizontal architecture. Shown is a schematic illustration of two sequencing unit cells laid out side-by-side and axisymmetric with respect to the trans well, i.e., the two sequencing unit cells are sharing the same trans well. A flowcell 170 of the nanopore sequencing device 110 is arranged between a bottom wafer 157 and a top wafer 167. The flowcell 170 may be filled with an electrolyte. Above the top wafer 167, a top portion 180 of the nanopore sequencing device 110 may be connected to external fluidic fixtures. Openings or holes 160 may be formed within the top wafer 167 to allow fluidic communications.

The nanopore sequencing device 110 includes a cis electrode 130 associated with a cis well 114. The nanopore sequencing device 110 further includes a trans electrode 134 associated with a trans well 116. In one example, the cis electrode 130 and the trans electrode 134 are arranged in an at least substantially horizontal direction with respect to the wafers. In other examples, the cis electrode and the trans electrode may be in any suitable orientation relative to each other and to the wafers. A divider 139 may separate the cis electrode 130/cis well 114 from the trans electrode 134/trans well 116. A middle well 115 is positioned between the cis well 114 and the trans well 116, and the cis well, middle well, and the trans well are oriented side by side horizontally.

The cis well 114 is connected to a nanopore 123, in which a first nanoscale opening is formed. In some embodiments, the nanopore 123 may be formed in a protein 118 disposed into a membrane 124. In some embodiments, the membrane 124 may be arranged vertically with respect to the wafers and the nanochannel, on a side of the cis well 114. The nanopore 123 provides a fluidic pathway for an electrolyte to pass between the cis well 114 and the middle well 115. The nanopore 123 fluidically communicates with a nanochannel 125 through the middle well 115. The nanochannel 125, in which a second nanoscale opening is formed, provides a fluidic/ionic/electric pathway for the electrolyte/current to pass between the middle well 115 and the trans well 116. The trans electrode 134 may be operably connected to a voltage supplier 111. The flowcell 170 includes the cis well 114, the trans well 116, a plurality of middle wells and their respective nanopores and nanochannels, all of which are in fluidic communication. A characteristic width of the middle well 115 may be about 5 μm, 10 μm, 20 μm, 30 μm, 40 μm, 50 μm, 60 μm, 70 μm, 80 μm, 90 μm, 100 μm, 150 μm, 200 μm, or any value therebetween. A characteristic depth of the middle well 115 may be about 5 μm, 10 μm, 20 μm, 30 μm, 40 μm, 50 μm, 60 μm, 70 μm, 80 μm, 90 μm, 100 μm, 150 μm, 200 μm, or any value therebetween. A characteristic width of the cis well or the trans well may be about 10 μm, 50 μm, 100 μm, 200 μm, 300 μm, 400 μm, 500 μm, 600 μm, 700 μm, 800 μm, 900 μm, 1 mm, 5 mm, 10 mm, or any value therebetween. The walls of the middle well 115 may be at least partly defined by wall structures 147, 159 and 149.

In some embodiments, the nanochannel 125 may be formed on the surface of the wafer horizontally or at least partially horizontally with respect to the wafers. In some embodiments, the nanochannel does not comprise a through-hole in the substrate. The width or diameter of the nanochannel 125 may be about 5 nm, 10 nm, 15 nm, 20 nm, 30 nm, 40 nm, 50 nm, 60 nm, 70 nm, 80 nm, 90 nm, 100 nm, 150 nm, 200 nm, or any value therebetween. The width of the nanochannel 125 may be adjusted by a deposited layer 137. The width of the nanochannel may be narrowed by the deposited layer by about 5%, 10%, 15%, 20%, 25%, 30%, 35%, 40%, 45%, 50%, 55%, 60%, 65%, 70%, 75%, 80%, 85%, 90%, or any value therebetween. In some embodiments, the nanochannel 125 may have a meandering, serpentine or tortuous path, in order to achieve a longer nanochannel path length while keeping the footprint small. A characteristic size of the nanochannel footprint (e.g., the length of the footprint) may be about 5 μm, 10 μm, 15 μm, 20 μm, 25 μm, 30 μm, 40 μm, 50 μm, 100 μm, 200 μm, 300 μm, 400 μm, 500 μm, or any value therebetween. A total path length of a nanochannel may be about 1.5, 2, 3, 4, 5, 6, 7, 8, 9, 10, 15, 20, 25, 30, 35, 40, 45, 50 times, or any value therebetween, of the characteristic size of the nanochannel footprint. A longer nanochannel path length may allow for a larger nanochannel resistance. The tortuous path may be a rectangular wave shape, a sine wave shape, a sawtooth shape, a zigzag shape, a spiral shape, or any combination thereof. The tortuous path may include a rectangular wave shape, a sine wave shape, a sawtooth shape, a zigzag shape, a spiral shape, or any combination thereof as a part of its shape.

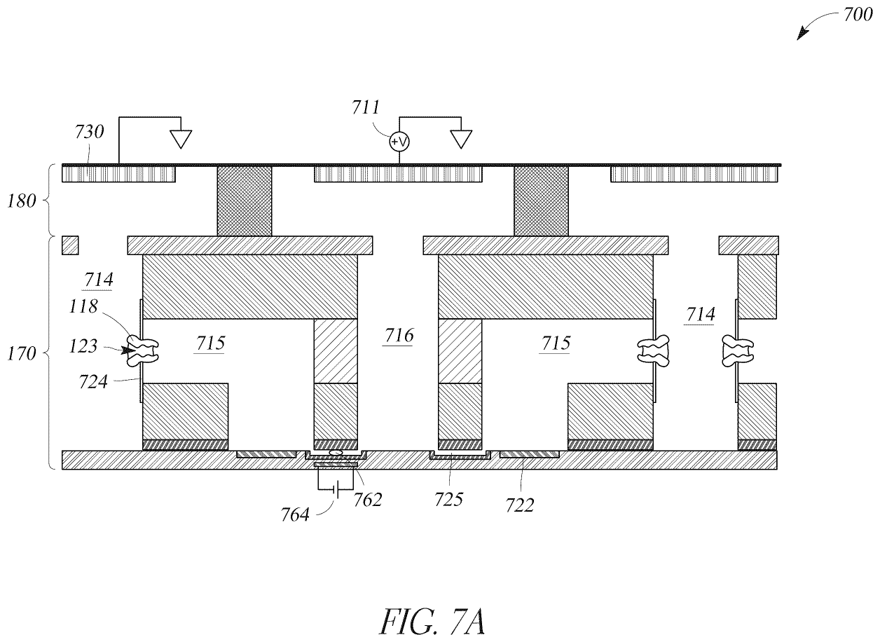

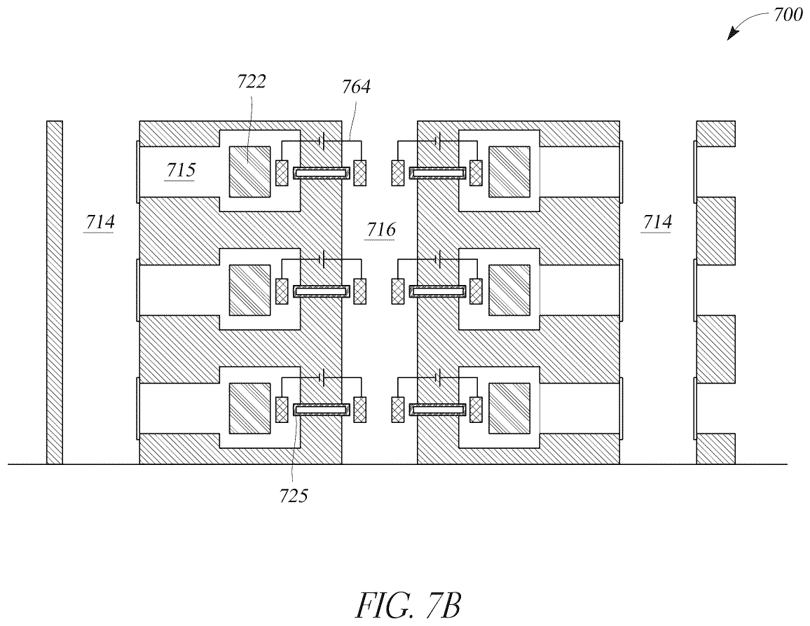

In some embodiments, a nanopore sequencing device, such as the device 110, may further include electronics or actuators arranged relatively underneath, above, to the side(s) of, and/or around some or all of the nanochannels to actively control liquid flow in some or all of the nanochannels. In some examples, micro-heaters (such as a resistive heater illustrate in FIG. 7A), optical transducers, pressure-based transducers, electromagnetic acoustic transducers may be included. For another example, ultrasound transducers may be used to generate pressure pulses in the liquid to eliminate bubbles or other unwanted debris from the nanochannels. Further methods of manipulating liquid flows can be found in Arango, Yulieth, et al. “Electro-actuated valves and self-vented channels enable programmable flow control and monitoring in capillary-driven microfluidics.” Science Advances 6.16 (2020): eaay8305, the disclosure of which is incorporated herein by reference.

In some embodiments, a nanopore sequencing device may further include electrodes to generate gas bubbles by electrolysis of the fluid in the nanochannels to block the liquid flow. For example, in the event of rupture and/or failure of the membrane and/or nanopore, gas bubbles can be generated to block ionic current flow of the non-performing sequencing unit cell so that the other performing cis/trans cells may continue to properly perform. The electrodes can create an electric field across the nanochannel and/or providing electron source/sink for electrolysis of the fluid within the electric field.

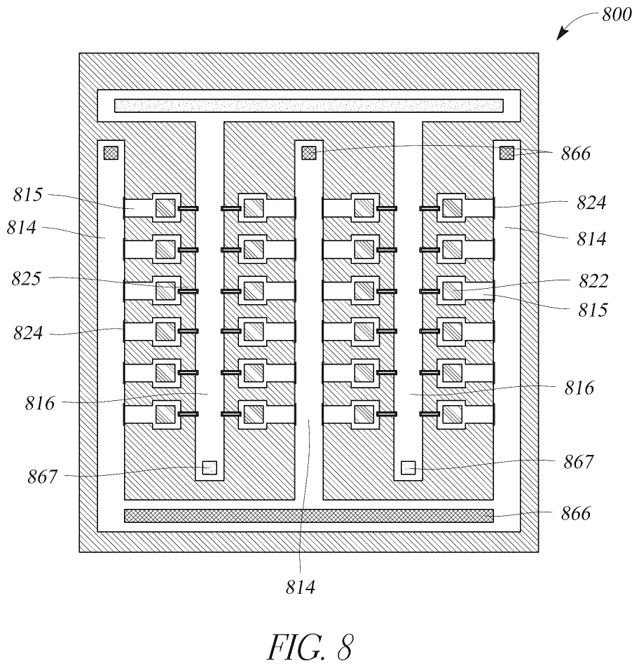

In some embodiments, the first and the second electrodes can be above and/or below the nanochannel. In some embodiments, the first and the second electrodes can be on each of the sides of the nanochannel. In some embodiments, the first electrode can be surrounding a first portion of the channel and the second electrode can be surrounding a second portion of the channel. In one example shown in FIG. 1A, the pair of electrodes 1001 and 1002 may be formed underneath the nanochannel 125. In another example shown in FIG. 8, the pair of electrodes 1001 and 1002 may be formed at outside of the nanochannel 125, at about the entrance and the exit of the nanochannel 125. FIG. 1B illustrates another example for generating water electrolysis. As shown in this figure, in some embodiments, a bubble can be generated on the bottom sensing electrode/FET as one of the electrode pair, and the CIS electrode and/or Trans electrode or another electrode can be utilized as the other electrode pair.

An electric field is created between the first and the second electrodes by having the electrodes at different potentials. For example, the voltage applied on the first electrode may range from +1 Volt to +2 Volt and the voltage applied on the second electrode may range from −1 Volt to −2 Volt, or vice versa. In some embodiments, one of the electrodes can be biased and other electrode can be grounded. In some embodiments, one of the electrodes can be biased and other electrode can be floating. In some embodiments, both of the electrodes can be biased but at different potentials. In some embodiments, the electrodes for generating water electrolysis may be non-reactive with the electrolyte. For example, the electrodes may be formed of platinum, iridium, ruthenium, palladium, tantalum, gold, TiN, or any combination thereof.

As exemplified in FIG. 1A, a sensing electrode 122 may be arranged in the bottom wafer (or the first substrate) 157 and may be exposed to the electrolyte in the middle well 115. The sensing electrode 122 may be used to detect the electrical potential of the electrolyte in the middle well and to transmit the detected signal to a voltage detector circuit or a field effect transistor. The voltage detector circuit or the field effect transistor may be external to the nanopore sequencing device 110, or may be arranged in the bottom wafer 157. The sensing electrode 122 may be made of corrosion-resistant metals with respect to the electrolyte. The sensing electrode 122 may be made of platinum, iridium, ruthenium, palladium, tantalum, gold, TiN, or any combination thereof. No electrochemical reaction may occur at the sensing electrode 122.

The membrane in a nanopore sequencing device may be formed from any suitable natural or synthetic material. In some embodiments, the membrane may be formed of a non-permeable or semi-permeable material. In an example shown in FIG. 1A, the membrane 124 is selected from the group consisting of a lipid and a biomimetic equivalent of a lipid. The nanopore in a nanopore sequencing device may be any of the biological nanopores, solid-state nanopores, hybrid nanopores, and synthetic nanopores described herein. I some embodiments, the nanopore may be a hollow defined by, for example: a polynucleotide structure, a polypeptide structure, or a solid-state structure, e.g., a carbon nanotube, which is disposed in the membrane. In some embodiments, the membrane may be a synthetic membrane (e.g., a solid-state membrane, one example of which is silicon nitride), and the nanopore is in a hollow extending through the membrane. In an example, the nanopore inner diameter ranges from about 0.5 nm to about 3 nm. In another example, the nanopore inner diameter ranges from about 1 nm to about 2 nm. In yet another example, the nanopore inner diameter ranges from about 1 nm to about 3 nm. The example ranges for the nanopore given above are intended to be the smallest diameter of the nanopore.

For example, as shown in FIG. 1A, a protein 118 having a hollow may be inserted into the membrane 124 directly, or the membrane may be formed around the protein. In an example, the protein may insert itself into a formed lipid bilayer membrane. For example, a protein in its monomeric form or polymeric form (e.g., an octamer) may insert itself into the lipid bilayer and assemble into a transmembrane pore. In another example, the protein may be added to a grounded side of a lipid bilayer at a desirable concentration where it will insert itself into the lipid bilayer. In still another example, the lipid bilayer may be formed across an aperture in a polytetrafluoroethylene (PTFE) film or any photo-patternable material (e.g., NIL resin), polyimide, silicon, or glass that is chemically stable and does not dissolve in the chemicals used for sequencing, and positioned between the cis well and the middle well. The protein may be added to the cis compartment, and may insert itself into the lipid bilayer at the area where the PTFE aperture is formed. In yet a further example, the protein may be tethered to a solid support (e.g., silicon, silicon oxide, quartz, indium tin oxide, gold, polymer, etc.). A tethering molecule, which may be part of the protein itself or may be attached to the protein, may attach the protein to the solid support. The attachment via the tethering molecule may be such that a single protein is immobilized between the cis well and the middle well. A lipid bilayer may then be formed around the protein.

The cis electrode that is used for nanopore sequencing devices as disclosed herein depends, at least in part, upon the redox couple in the electrolyte. As examples, the cis electrode may be gold (Au), platinum (Pt), carbon (C) (e.g., graphite, diamond, etc.), palladium (Pd), silver (Ag), copper (Cu), or the like. In an example, the cis electrode may be a silver/silver chloride (Ag/AgCl) electrode. In one example, the cis well is capable of maintaining the electrolyte in contact with the first nanoscale opening. In some examples, the cis well may be in contact with an array of nanopores, and thus is capable of maintaining the electrolyte in contact with each of the nanopores in the array.

The trans electrode that is used for nanopore sequencing devices as disclosed herein depends, at least in part, upon the redox couple in the electrolyte. As examples, the trans electrode may be gold (Au), platinum (Pt), carbon (C) (e.g., graphite, diamond, etc.), palladium (Pd), silver (Ag), copper (Cu), or the like. In an example, the trans electrode may be a silver/silver chloride (Ag/AgCl) electrode.

In some examples, the relevant electrochemical half-reactions at the electrodes for a Ag/AgCl electrode in NaCl or KCl solution, are:

Cis (cathode): AgCl+e⁻→Ag⁰+Cl⁻; and

Trans (anode): Ag⁰+Cl⁻→AgCl+e⁻.

For every unit charge of current, one Cl atom is consumed at the trans electrode. Though the discussion above is in terms of an Ag/AgCl electrode in NaCl or KCl solution, it is to be understood that any electrode/electrolyte pair that may be used to pass the current may apply.

In use, an electrolyte may be filled into the cis well, the middle well, and the trans well. In alternative examples, the electrolyte in the cis well, the middle well, and the trans well may be different. The electrolyte may be any electrolyte that is capable of dissociating into counter ions (a cation and its associated anion). As examples, the electrolyte may be an electrolyte that is capable of dissociating into a potassium cation (K⁺) or a sodium cation (Na⁺). This type of electrolyte includes a potassium cation and an associated anion, or a sodium cation and an associated anion, or combinations thereof. Examples of potassium-containing electrolytes include potassium chloride (KCl), potassium ferricyanide (K₃[Fc(CN)₆]·3H₂O or K₄[Fe(CN)₆]·3H₂O), or other potassium-containing electrolytes (e.g., bicarbonate (KHCO₃) or phosphates (e.g., KH₂PO₄, K₂HPO₄, K₃PO₄). Examples of sodium-containing electrolytes include sodium chloride (NaCl) or other sodium-containing electrolytes, such as sodium bicarbonate (NaHCO₃), sodium phosphates (e.g., NaH₂PO₄, Na₂HPO₄ or Na₃PO₄). As another example, the electrolyte may be any electrolyte that is capable of dissociating into a ruthenium-containing cation (e.g., ruthenium hexamine, such as [Ru(NH₃)₆]²⁺ or [Ru(NH₃)₆]³⁺). Electrolytes that are capable of dissociating into a lithium cation (Li⁺), a rubidium cation (Rb⁺), a magnesium cation (Mg⁺), or a calcium cation (Ca⁺) may also be used.

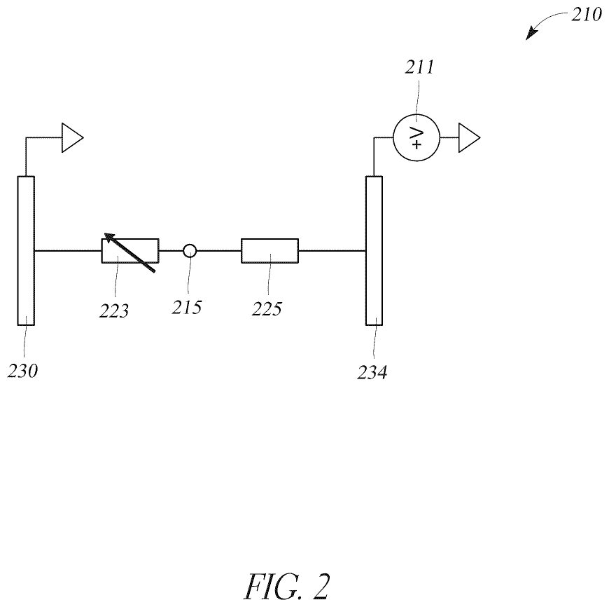

FIG. 2 shows an equivalent circuit diagram 210 of a nanopore sequencing device described herein. To use the nanopore sequencing device, an electrolyte is introduced into each of the cis well, the trans well, and the middle well. A voltage difference V is applied between the cis electrode (indicated as node 230 in FIG. 2) and the trans electrode (indicated as node 234 in FIG. 2) by the voltage supplier (indicated as feature 111 in FIG. 1A and feature 211 in FIG. 2). During operation, the range of applied voltages can be selected from about −0.1 mV to upwards of about 0.1 mV, from about −0.5 mV to upwards of about 0.5 mV, from about −1 mV to upwards of about 1 mV, from about −1.5 mV to upwards of about 1.5 mV, from about −2.0 mV to upwards of about 2.0 mV, from about −3.0 mV to upwards of about 3.0 mV, from about −5.0 mV to upwards of about 5.0 mV, from about −0.1 V to upwards of about 0.1 V, from about −0.5 V to upwards of about 0.5 V, from about −1 V to upwards of about 1 V, from about −1.5 V to upwards of about 1.5 V, from about −2.0 V to upwards of about 2.0 V, from about −3.0 V to upwards of about 3.0 V, or from about −5.0 V to upwards of about 5.0 V. In some instances, the voltage polarity may be applied such that a negatively charged nucleic acid is electrophoretically driven towards the trans electrode. In some instances, the voltage polarity may be applied such that a positively charge protein is electrophoretically driven towards the trans electrode. In some instances, the voltage can be reduced, or the polarity reversed, to facilitate appropriate function of the device.

In some examples, a polynucleotide is driven through the nanopore. During a nanopore sequencing operation, the application of the voltage difference V across the cis electrode 130 and the trans electrode may force the translocation of a nucleotide through the nanopore along with the anions carrying charges. Depending upon the polarity of the voltage difference, the nucleotide may be transported from the cis well to middle well, or from the middle well to the cis well. As the nucleotide transits through the nanopore, the current across the membrane may change due to nucleobase-dependent blockage of the nanopore constriction.

In alternative examples, the polynucleotide does not pass through the nanopore, but tagged nucleotides are incorporated by a polymerase acting on the polynucleotide. In certain embodiments, a single-stranded polynucleotide, a double-stranded polynucleotide, tags or labels of incorporated nucleotides, or other representatives of the incorporated nucleotides, and any combination thereof may pass through the nanopore. In certain embodiments, tags or labels of incorporated nucleotides may be separated or dissociated from the polynucleotide, and such tags or labels may pass through the nanopore with or without the polynucleotide passing through the nanopore. Examples are not limited to how the polynucleotide communicates with the nanopore to cause signal generation in the nanopore sequencing device.

An electrical resistance Rₚ of the nanopore (indicated as a resistor 223 in FIG. 2) varies in response to the identity of one or more nucleobases near or at the nanopore, for example, while a nucleotide of the polynucleotide passes through the nanopore, or while a tagged nucleotide is being incorporated by a polymerase acting on the polynucleotide, thus the different tags of the tagged nucleotides change the resistance of the nanopore. In some examples, as the polynucleotide enters the constriction of the nanopore, the resistance Rₚ is modulated based on the identity of the bases in the polynucleotide. In other examples, the resistance Rₚ is modulated based on the identity of a tag in the nanopore constriction, while the corresponding tagged nucleotide is being incorporated by a polymerase acting on the polynucleotide. In some examples, the resistance of the nanopore, Rₚ, changes as a function of the nucleobase at or near the nanopore and may range from about 0.5 to about 5 giga-ohm (GΩ). The resistance Rₚ may be relatively large and may vary by 30-40% as a function of different polynucleotide bases at or near the nanopore. In some examples, the resistance Rₚ may vary by between about 0.001% to about 1%, about 1% to about 5%, about 5% to about 20%, about 20% to about 40%, about 40% to about 60%, or 60% to about 100%.

In some examples, the nanochannel has a fixed, or substantially fixed electrical resistance Re (indicated as a resistor 225 in FIG. 2). The resistance Re of the nanochannel is not modulated by the nucleobase of the polynucleotide at or near the nanopore. In some examples, the resistance of the nanochannel, Re, may be about 1 to 5 giga-ohm (GΩ).

The equivalent circuit 210 shown in FIG. 2 is a voltage divider, where the electrical potential of point 215 is the potential of the electrolyte in the middle well. In certain embodiments, the equivalent circuit of the nanopore sequencing device satisfies the following equations:

The potential V_{M} at point 215 given by

$${V}_{M} = D V \qquad ( 1 )$$

where D is the voltage divider ratio

$$D = \frac{{R}_{p}}{{R}_{c} + {R}_{p}} \qquad ( 2 )$$

and V is the cis-trans bias.

The electrical potential V_{M} of the electrolyte in the middle well (indicated as the voltage divider point 215 in FIG. 2) varies in response to the variation in electrical resistance Rₚ of the nanopore. Therefore, measuring the electrical potential at the voltage divider point 215 as the resistance Rₚ changes permits determination of the resistance Rₚ, and such information can be used to identify the nucleobases in the polynucleotide. In some examples, measuring the electrical potential at the voltage divider point 215 may be achieved by coupling the sensing electrode to a voltage detector. In some examples, measuring the electrical potential at the voltage divider point 215 may be achieved by coupling an FET sensor to the middle well. In one embodiment, the FET gate is coupled to the sensing electrode, such that the electrical potential of the voltage divider point 215 acts as the FET gate potential and establishes the FET operating point. Examples of measuring the response of the FET include measuring a source-drain current or measuring a potential at the source and/or drain. Additionally, a resistance of the FET channel can be measured to identify the nucleobase in the polynucleotide.

A method of using a nanopore sequencing device may include introducing an electrolyte into each of the cis well, the trans well, and the middle well. After introducing the electrolyte, the method may include providing a polynucleotide to be sequenced into the cis well. After providing the polynucleotide, the method may include applying a voltage bias between the cis electrode and the trans electrode. In some embodiments, the voltage bias may drive the polynucleotide from the cis well to the middle well, through the nanopore. As the polynucleotide passes through the nanopore, the electrical resistance of the nanopore varies in response to an identity of nucleobases in the polynucleotide at the nanopore. In alternative embodiments, the polynucleotide does not pass through the nanopore, but tags or labels of nucleotides being incorporated by a polymerase acting on the polynucleotide may pass through the nanopore or may temporarily reside in the nanopore. Thus, the electrical resistance of the nanopore varies in response to an identity of the nucleotide being incorporated, which is complementary to the identity of a base in the polynucleotide. As a result, the potential (V_{M}) of the electrolyte in the middle well varies with the identities of bases in the polynucleotide. The potential (V_{M}) may be measured from the sensing electrode. The potential (V_{M}) may be the gate voltage applied to a FET, which modulates the conductivity of the FET channel. Therefore, measurements of the response of the FET can determine the identity of the bases in the polynucleotide.

In some embodiments of a nanopore sequencing device, one or more trans wells are fluidically connected to one or more cis wells by a plurality of middle wells and the respective nanopores and nanochannels. In various embodiments, the one or more trans wells may or may not be interconnected. In various embodiments, the one or more cis wells may or may not be interconnected. Each of the one or more trans wells may be associated with a respective trans electrode. In various embodiments, the trans electrodes may or may not be operably connected to each other. Each of the one or more cis wells may be associated with a respective cis electrode. In various embodiments, the cis electrodes may or may not be operably connected to each other. In some embodiments, at least a group of trans wells are interconnected, at least a group of cis wells are interconnected, at least a group of trans electrodes are operably interconnected, at least a group of cis electrodes are operably interconnected, or any combination thereof.

In embodiments where a plurality of sequencing unit cells forms an array on a chip, each of the plurality of the sequencing unit cells in the array may share a common cis electrode and a common trans electrode. In another example, each of the plurality of the sequencing unit cells shares a common cis electrode, but has a distinct trans electrode. In yet another example, each of the plurality of the sequencing unit cells has a distinct cis electrode and a distinct trans electrode. In still another example, each of the plurality of sequencing unit cells has a distinct cis electrode and shares a common trans electrode. In some embodiments, at least a group of sequencing unit cells in the array may share a common cis electrode and a common trans electrode. In some embodiments, at least a group of sequencing unit cells shares a common cis electrode, but each member of the group has a distinct trans electrode. In some embodiments, at least a group of sequencing unit cells shares a common trans electrode, but each member of the group has a distinct cis electrode.

FIG. 3A is a cross-sectional top view of the nanopore sequencing device of FIG. 1A, showing an array 300A of sequencing unit cells. An array of straight nanochannels connect an array of middle wells to a trans well. For example, the cis well 314A connects to a sequencing unit cell which includes a membrane 324A with a nanopore disposed in it (not shown), a middle well 315A, and a straight nanochannel 325A. The sensing electrode 322A at the bottom of the middle well 315A may be used to detect the electrical potential of the electrolyte in the middle well of this sequencing unit cell. The sequencing unit cell then connects to the trans well 316A. The effective width of the straight nanochannel 325A is narrowed by a deposited layer 337A. Also shown in FIG. 3A is the wall structure 359A which separates the individual sequencing unit cells.

FIG. 3B is a cross-sectional top view of the nanopore sequencing device of FIG. 1A having an alternative nanochannel structure. FIG. 3B shows an array 300B of sequencing unit cells where an array of meandering/serpentine/tortuous nanochannels connect an array of middle wells to a trans well. For example, the cis well 314B connects to a sequencing unit cell which includes a membrane 324B with a nanopore disposed in it (not shown), a middle well 315B, and a meandering nanochannel 325B. The sensing electrode 322B at the bottom of the middle well 315B may be used to detect the electrical potential of the electrolyte in the middle well of this sequencing unit cell. The sequencing unit cell then connects to the trans well 316B. The effective width of the meandering nanochannel 325B is narrowed by a deposited layer 337B. Also shown in FIG. 3B is the wall structure 359B which separates the individual sequencing unit cells. In the example shown in FIG. 3B, the meandering nanochannel 325B has rectangular wave shape. Alternatively, the meandering nanochannel 325B may have a sine wave shape, a sawtooth shape, a zigzag shape, a spiral shape, or any combination thereof.

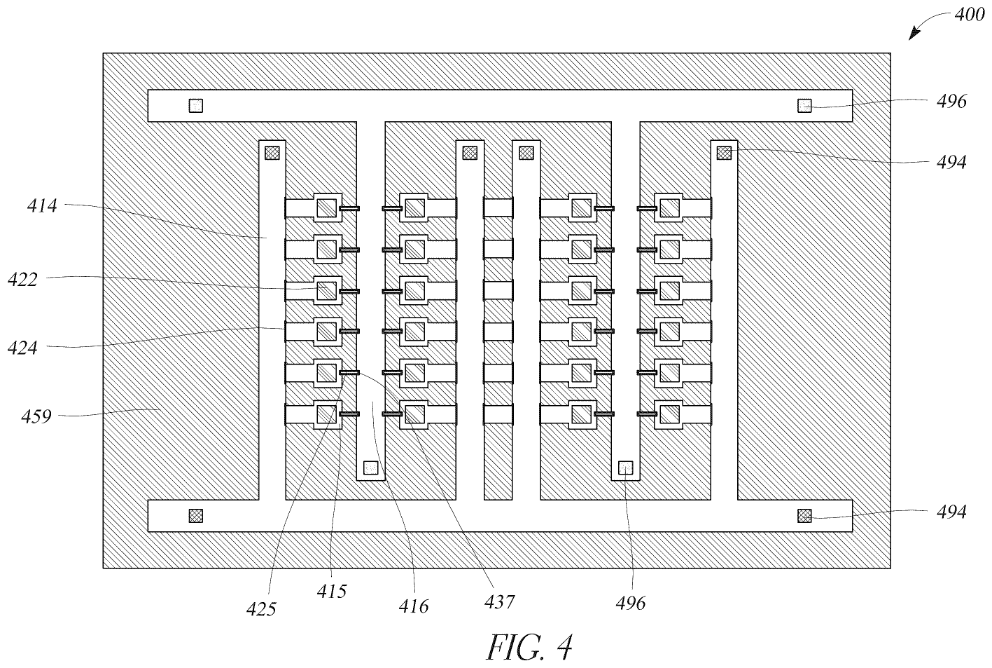

FIG. 4 is a cross-sectional top view of an example sequencing system 400 including the nanopore sequencing devices of FIG. 1A and inlet/outlet holes which allow fluidic and electric contact to cis/trans wells. For example, the cis well 414 connects to an array of sequencing unit cells. A sequencing unit cell includes a membrane (an example is the feature labeled 424), a middle well (an example is the feature labeled 415), and a nanochannel (an example is the feature labeled 425). The membrane includes a nanopore disposed in it (but not shown). A sensing electrode (an example is the feature labeled 422) at the bottom of a middle well 415 may be used to detect the electrical potential of the electrolyte in this middle well. The sequencing unit cells connects to the trans well 416. The effective width of a nanochannel is narrowed by a deposited layer (an example is the feature labeled 437). Also shown in FIG. 4 is the wall structure 459 which separates the individual sequencing unit cells. To allow fluidic or electrical contact and material exchange with external fluidic fixtures, the cis well 414 are connected with cis inlets/outlets 494 and the trans well 416 are connected with cis trans/outlets 496.

FIG. 8 is a cross-sectional top view of another example sequencing system 800 including the nanopore sequencing devices of FIG. 1A and inlet/outlet holes which allow fluidic and electric contact to cis well 866 and/or trans well 867. In some embodiments, the array comprises a single shared or common cis well 814. In some embodiments, the array comprises a shared or common trans well 816. A sequencing unit cell includes a membrane (an example is the feature labeled 824), a middle well (an example is the feature labeled 815), and a nanochannel (an example is the feature labeled 825). In some embodiments, the nanochannel does not comprise a through-hole in the substrate. The membrane includes a nanopore disposed in it (but not shown). A sensing electrode (an example is the feature labeled 822) at the bottom of a middle well 815 may be used to detect the electrical potential of the electrolyte in this middle well. The sequencing unit cells connects to the trans well 816. To allow fluidic or electrical contact and material exchange with external fluidic fixtures, the cis well 814 are connected with cis inlets/outlets 866 and the trans well 816 are connected with trans inlets/outlets 867.

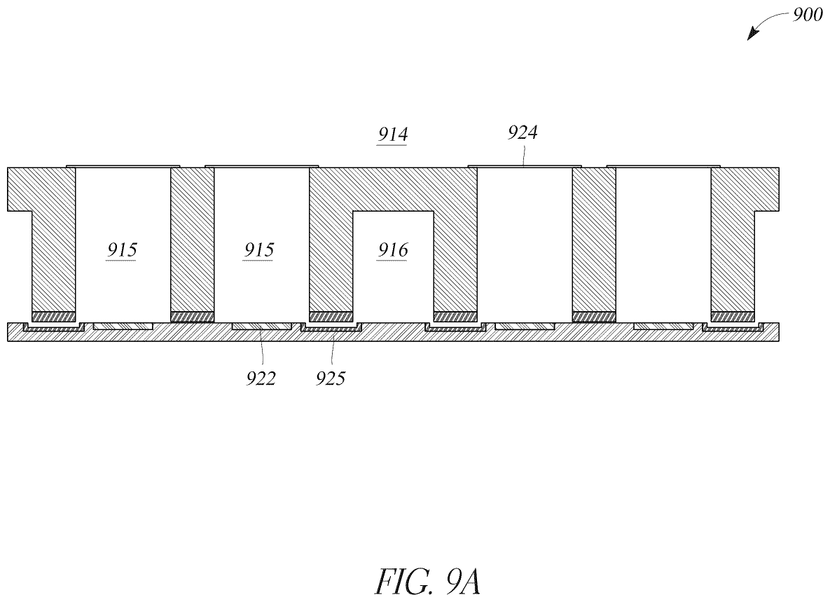

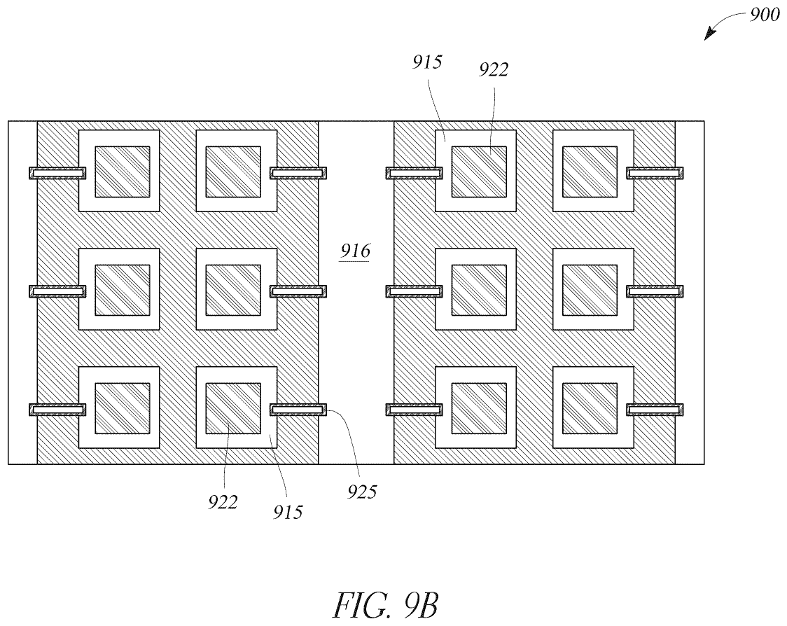

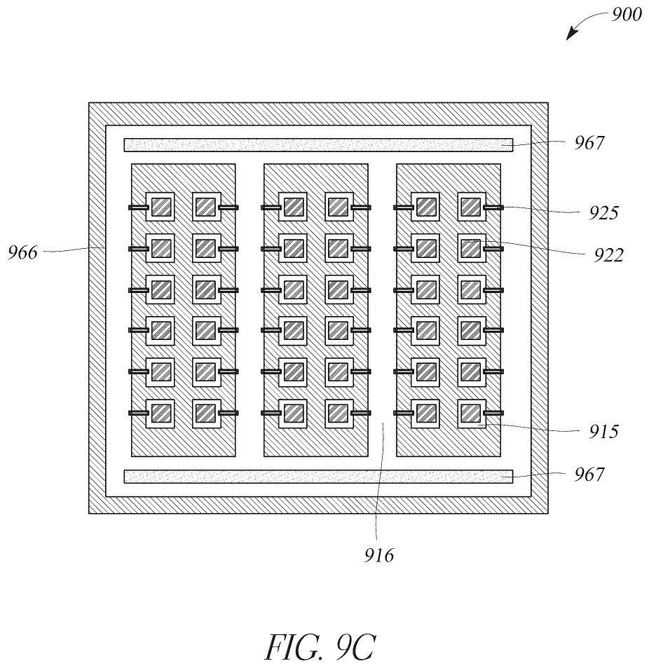

In some embodiments, a nanopore sequencing device may have a cis well or cis channel position over the middle well. FIG. 9A shows a cross-sectional side view of an example nanopore sequencing device with the cis well on top of the device as part of the fluidic fixture and not integrated into the device. As shown in FIGS. 9A, 9B, and 9C, in embodiments 900, the cis wells 914 are positioned so that membranes 924 are formed horizontally parallel to the horizontal nanochannel 925. In some embodiments, the nanochannel does not comprise a through-hole in the substrate. For example, in some embodiments, the nanochannel 925 is etched into a substrate. In some embodiments, the substrate comprises a semiconductor wafer.

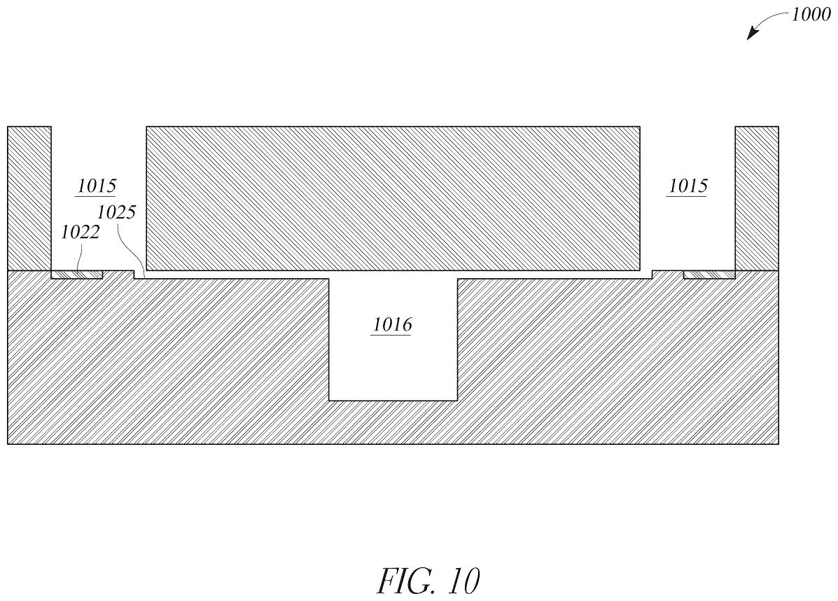

In some embodiment, the trans well may be formed in the dielectric layer of the first substrate, resulting in a trans well that is position vertically with respect to the middle well. FIG. 10 shows a cross-sectional side view of an example nanopore sequencing device with a common trans well 1016 fluidically connected to two middle wells 1015 through respective nanochannels 1025. The trans well 1016 is formed in the substrate, and is thus below the substrate surface. The middle well 1015 is form in the patterned layer and is thus above the substrate. The nanochannel 1025 is form on the substrate surface, and it does not contain a through-hole in the substrate. In some embodiments, the cis well (not shown) may be side-by-side with and next to the middle well 1015, similar to the embodiment illustrated in FIG. 1A. In other embodiments, the cis well may be positioned over the middle well 1016, similar to the embodiment illustrated in FIG. 9A.

In a chip with an array of nanopore sequencing devices, there may be one common cis well and one common trans well communicating with a portion or all of the nanopore sequencing unit cells within the array in the chip. However, it should be understood that an array of the nanopore devices may also include several cis wells that are fluidically isolated from one another and are fluidically connected to respective one or more trans wells fluidically isolated from one another. Multiple cis wells may be desirable, for example, in order to enable the measurement of multiple samples on a single chip. In some embodiments, a chip with an array of nanopore sequencing devices comprises one common cis electrode, one common trans electrode, one common cis well, one common trans well, and a plurality of nanopore sequencing devices, where each nanopore sequencing device can separately measure a single molecule of polynucleotide. In other embodiments, the chip with an array of nanopore sequencing devices comprises one common cis well, a plurality of trans wells, and a plurality of nanopore sequencing devices, where each nanopore sequencing device can be individually addressable with individual trans electrodes. In other embodiments, the chip with an array of nanopore sequencing devices comprises a plurality of cis wells, a plurality of trans wells, and a plurality of nanopore sequencing devices, where each nanopore sequencing device can be individually addressable with individual trans electrodes.

### Additional Embodiments

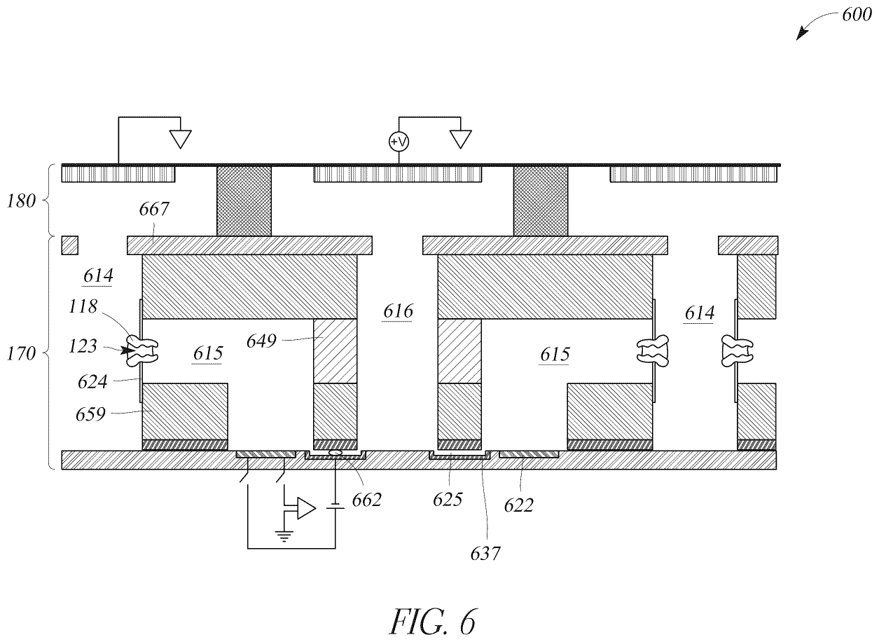

FIG. 6 illustrates yet another example nanopore sequencing device which can generate bubbles via water electrolysis. Shown is an illustration of two sequencing unit cells laid out side-by-side and sharing the same trans well 616. A flowcell of the nanopore sequencing device is arranged between a first substrate and a second substrate. The flowcell may be filled with an electrolyte.

Above the second substrate, a top portion of the nanopore sequencing device may be connected to external fluidic fixtures. The cis well 614 is connected to a nanopore 623, in which a first nanoscale opening is formed. In some embodiments, the nanopore 623 may be formed in a protein 618 disposed into a membrane 624. In some embodiments, the membrane 624 may be arranged vertically with respect to the first and second substrates, on a side of the cis well 614. The nanopore 623 provides a fluidic pathway for an electrolyte to pass between the cis well 614 and the middle well 615. The nanopore 623 fluidically communicates with a nanochannel 625 through the middle well 615. The nanochannel 625, in which a second nanoscale opening is formed, provides a fluidic/ionic/electric pathway for the electrolyte/current to pass between the middle well 615 and the trans well 616. The trans electrode may be operably connected to a voltage supplier. The flowcell includes the cis well 614, the trans well 616, a plurality of middle wells 615 and their respective nanopores 623 and nanochannels 625, all of which are in fluidic communication.

The walls of the middle well 615 may be at least partly defined by wall structures 659. In some embodiments, the device may further include electrodes to generate gas bubbles 662 by electrolysis of the fluid in the nanochannels to block the liquid flow. For example, in the event of rupture and/or failure of the membrane and/or nanopore, gas bubbles can be generated to block ionic current flow of the non-performing sequencing unit cell so that the other performing cis/trans cells may continue to properly perform.

A sensing electrode 622 may be arranged in the first substrate and may be exposed to the electrolyte in the middle well 615. The sensing electrode 622 may be used to detect the electrical potential of the electrolyte in the middle well and to transmit the detected signal to a voltage detector circuit or a field effect transistor. In some embodiments, the bottom sensing electrode/FET 622 is used as one of the electrode pair, and the horizontal nanochannel 625 which is metallized is used as the other electrode for electrolysis. Two switches may be added: (1) a first switch added to enable the bottom electrode/FET in a sensing mode and (2) a second switch added to enable the bottom electrode plus horizontal channel electrode in electrolysis mode.

FIG. 7A is a cross-sectional side view of an example nanopore sequencing device 700. The nanopore sequencing device 700 has the same structure as the embodiment shown in FIG. 6, except the bubble generating element is a micro heater or a resistive heater 764. In FIG. 7A, the heating element 764 is underneath the nanochannel. The heating element may generate a bubble 762 within the nanochannel 725.

FIG. 7B is a top view of another example nanopore sequencing device 700. The nanopore sequencing device 700 has the same structure as the embodiment shown in FIG. 6, except two electrolysis electrodes 770 are located near the openings of the nanochannel 725. The electrodes can generate a bubble within the nanochannel.

### Example Process of Manufacturing a Nanopore Sequencing Device

Some aspects of the present disclosure are directed to methods of manufacturing a nanopore sequencing device. In some embodiments, the method comprises: providing a first substrate comprising a dielectric layer and at least one sensing electrode on a surface of the first substrate; forming at least one nanochannel on the surface of the first substrate; depositing a sacrificial filler layer in the at least one nanochannel; depositing a capping layer over the sacrificial filler layer; patterning the capping layer to expose the at least one sensing electrode and openings to the at least one nanochannel; and removing the sacrificial filler layer, thereby opening the at least one nanochannel. In some embodiments, the method of manufacturing the nanopore sequencing device comprises providing a first substrate comprising a dielectric layer and at least one sensing electrode on a surface of the first substrate; forming a trans well in the dielectric layer; and forming at least one nanochannel on the surface of the first substrate between the trans well and the at least one sensing electrode. In some embodiments, the nanochannel does not comprise a through-hole in the substrate.

FIG. 5A to FIG. 5L illustrate an example fabrication process flow of manufacturing a nanopore sequencing device as disclosed herein.

In the process step shown in FIG. 5A, a first substrate comprising a dielectric layer and at least one sensing electrode is provided. The first substrate includes a CMOS wafer 557 formed of Si substrate manufactured with an integrated circuit, for example a sensing electrode 522 formed of Ru or TiN. The sensing electrode 522 may be connected to a voltage detector circuit, a FET sensor or an amplifier 5221. At least one of the electrodes for electrolysis, 5001 or 5002, may also be provided in this step. In alternative embodiments, flexible substrates such as ultrathin glass, metal foil, and plastic (polymer) films may be used for the bottom wafer, which may be manufactured with flexible electronics made with organic or carbon-based transistors.

In the process step shown in FIG. 5B, definition and formation of the path of the nanochannels 525 may be performed by lithography and etching techniques.

In the optional process step shown in FIG. 5C, reduction of the nanochannel widths may be achieved by forming the deposited layer 537 via conformal oxide/nitride layer deposition.

In the process step shown in FIG. 5D, deposition of a sacrificial filler layer 5001 may be performed to fill the nanochannels with sacrificial material. The sacrificial layer 5001 may be formed of Ti or Al.

In the process step shown in FIG. 5E, planarization of the sacrificial layer may be performed by means of polishing. The sensing electrodes 522 is exposed, while the nanochannel path is filled with the sacrificial material. FIG. 5F shows a cross-sectional top view of the nanochannels with the deposited sacrificial material and exposed sensing electrodes 522.

In the process step shown in FIG. 5G, passivation of the wafer surface is performed by deposition of a capping layer 547, which may be formed of oxide/nitride. A portion of the capping layer 547 may provide the base of the wall structure 147 in FIG. 1A. In some embodiments, the passivation may also provide a hydrophilic cap for the nanochannels.

In the process step shown in FIG. 5H, the capping layer 547 is patterned to expose the sensing electrode and the openings of the nanochannels to the middle and trans wells (yet to be formed). The nanochannels are now formed with the capping layer closing the top of the nanochannel path and having the two openings form on each end of the nanochannel path. This step may be performed by lithography and etching techniques.

The process steps shown in FIGS. 5G and 5H may be optional. If the capping layer is not deposited after depositing the sacrificial material in the nanochannel path, the process step shown in FIG. 5I removal of the sacrificial material is performed after the process step shown in FIG. 5K.

In the process step shown in FIG. 5I, removal of the sacrificial material in the nanochannels may be achieved by wet etch techniques. Once the sacrificial material is removed, the nanochannels are opened and will allow fluid communication between the middle well and the trans well to be formed in a later step.

In the process step shown in FIG. 5J, deposition of a thick patterning material layer 559 over the surface of the wafer is performed. A portion of the patterning material layer 559 may provide the wall structure 159 in FIG. 1A. The patterning material layer 559 may be formed of SU-8 photoresist or nanoimprint lithography (NIL) resins, polyimide, any kind of photo-patternable thick (spin-coated or laminated) resists such as TMMF, TMMR, silicones such as PDMS, thermoplastics such as PMMA, COC, PC (polycarbonate), or any suitable dielectric material.

In the process step shown in FIG. 5K, patterning and etching of the patterning material layer by photolithography or nanoimprint lithography methods may form a first patterned layer which in part defines the middle well and the trans well, such as 515 and 516, respectively. In some embodiments, the first patterned layer may also in part define the cis wall, such as 514. In some embodiments, the trans well comprises a common trans well.

In the process step shown in FIG. 5L, a complementary second substrate is provided. The second substrate includes a top wafer 567 and a second patterned layer of SU-8 or NIL resins. The top wafer 567 may further include fluidic inlet/outlet holes, and a patterned adhesive layer 549 formed of, e.g., photocurable resins, such as SU-8 or benzocyclobutene (BCB), or other suitable polymers, spin-on glasses, resists, and polyimides, PDMS, fusion bonding or covalent bonding of SiO₂ surfaces, COC, methyl acrylic adhesive (see U.S. Pat. No. 20200009556A1, which is incorporated herein by reference). The patterned adhesive layer 549 may provide the wall structure 149 in FIG. 1A. The first patterned layer of the bottom wafer is aligned with the second patterned layer of the top wafer, and wafer bonding of the first patterned layer with the second patterned layer is performed via the adhesive layer 549. After wafer bonding, the cis, middle and trans wells are formed between the bottom wafer and the top wafer.

In the process step shown in FIG. 5M, membranes 524 may be introduced between the cis wells and middle wells and may be arranged vertically. The membrane has a nanopore disposed in it to provide fluid communication between the cis well and the middle well. In some embodiments, proteins such as MspA may be deposited into the lipid membranes to form nanopores through the membranes.

In some embodiments, the process step shown in FIG. 5L may not be needed. For example, for embodiments where the cis well is over the middle well shown in FIG. 9A, a second substrate may not be required in some instances. In some embodiments, membranes 924 may be deposited horizontally over the middle well 915, separating the middle well 915 and the cis well 914.

In some embodiments, a nanopore sequencing device as shown in FIG. 10 may be made by the following steps. Similar to the method described above, a first substrate including a dielectric layer and at least one sensing electrode 1022 on the surface is provided. The first substrate in these embodiments may have a thicker dielectric layer. The trans well 1016 is formed by patterning and etching into the dielectric layer of the first substrate. Thus the trans well is below the surface of the first substrate.

Next a nanochannel 1025 is formed on the surface of the first substrate between the trans well 1016 and the sensing electrode 1025, which will provide fluid communication between the trans well 1016 and the middle well 1015 that will be formed at a later step. A patterning material layer is then disposed over the first substrate. For example, the patterning material layer may be a dry film photoresist that is laminated over the substrate. The dry film photoresist may be any suitable photoresist material, including but not limited to TMMF and SU8. The patterning material layer is subsequently patterned to form a patterned layer that includes a middle well 1015 positioned above the sensing electrode 1022 as described above.

In some embodiments, the cis well may be formed in the patterned layer, resulting in embodiments where the cis well and the middle well being positioned side-by-side. In some embodiments, the cis well may be positioned over the middle well similar to the embodiment shown in FIG. 9A.

### Additional Notes

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such value or sub-range were explicitly recited. For example, a range from about 2 nm to about 20 nm should be interpreted to include not only the explicitly recited limits of from about 2 nm to about 20 nm, but also to include individual values, such as about 3.5 nm, about 8 nm, about 18.2 nm, etc., and sub-ranges, such as from about 5 nm to about 10 nm, etc. Furthermore, when “about” and/or “substantially” are/is utilized to describe a value, this is meant to encompass minor variations (up to +/−10%) from the stated value.

While several examples have been described in detail, it is to be understood that the disclosed examples may be modified. Therefore, the foregoing description is to be considered non-limiting.

While certain examples have been described, these examples have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure.

Features, materials, characteristics, or groups described in conjunction with a particular aspect, or example are to be understood to be applicable to any other aspect or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some examples, the actual steps taken in the processes illustrated and/or disclosed may differ from those shown in the figures. Depending on the example, certain of the steps described above may be removed or others may be added. Furthermore, the features and attributes of the specific examples disclosed above may be combined in different ways to form additional examples, all of which fall within the scope of the present disclosure. Also, the separation of various system components in the implementations described above should not be understood as requiring such separation in all implementations, and it should be understood that the described components and systems can generally be integrated together in a single product or packaged into multiple products. For example, any of the components for an energy storage system described herein can be provided separately, or integrated together (e.g., packaged together, or attached together) to form an energy storage system.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

The scope of the present disclosure is not intended to be limited by the specific disclosures of preferred examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A nanopore sequencing device, comprising:
a substrate comprising a dielectric layer and at least one sensing electrode on a surface of the dielectric layer;
a cis well associated with a cis electrode;
a trans well associated with a trans electrode;
a middle well associated with the sensing electrode and positioned on the substrate, wherein the middle well is positioned on the substrate and in fluid communication with the cis well and the trans well;
a nanopore fluidically connecting the cis well and the middle well; and
a nanochannel fluidically connecting the middle well and the trans well, wherein the nanochannel is formed on the surface of the substrate.

2. The nanopore sequencing device of claim 1, wherein the nanochannel does not comprise a through-hole in the substrate.

3. The nanopore sequencing device of claim 1, wherein the nanopore is positioned in and through a membrane separating the cis well and the middle well.

4. The nanopore sequencing device of claim 3, wherein the membrane is formed of lipid, silicon, graphene, a solid-state material, a synthetic material, a biomimetic equivalent of lipid, or any combination thereof.

5. The nanopore sequencing device of claim 3, wherein the nanopore is a hollow in a structure formed of one or more polynucleotides, one or more polypeptides, one or more types of biopolymers, one or more carbon nanotubes, one or more types of solid-state materials, or any combination thereof disposed in the membrane.

6. The nanopore sequencing device of claim 1, wherein the nanopore comprises biologically derived material.

7. The nanopore sequencing device of claim 6, wherein the nanopore comprises a porin.

8. The nanopore sequencing device of claim 1, wherein the nanopore comprises non-biologically derived material.

9. The nanopore sequencing device of claim 1, wherein at least the cis well or the trans well is positioned horizontally side-by-side with the middle well.

10. The nanopore sequencing device of claim 9, wherein both the cis well and the trans well are positioned horizontally side-by-side with the middle well.

11. The nanopore sequencing device of claim 9, wherein the cis well is positioned horizontally side-by-side with the middle well, and the trans well is positioned vertically adjacent to the middle well.

12. The nanopore sequencing device of claim 9, wherein the trans well is positioned horizontally side-by-side with the middle well, and the cis well is positioned vertically adjacent to the middle well.

13. The nanopore sequencing device of claim 1, wherein the middle well has a characteristic width of about 5 μm to about 200 μm.

14. The nanopore sequencing device of claim 1, wherein the middle well has a characteristic depth of about 5 μm to about 200 μm.

15. The nanopore sequencing device of claim 1, wherein the cis well has a characteristic width of about 10 μm to about 10 mm.

16. The nanopore sequencing device of claim 1, wherein the trans well has a characteristic width of about 10 μm to about 10 mm.

17. The nanopore sequencing device of claim 1, wherein the nanochannel has a tortuous path.

18. The nanopore sequencing device of claim 17, wherein the tortuous path comprises a rectangular wave shape, a sine wave shape, a sawtooth shape, a zigzag shape, a spiral shape, or any combination thereof.

19. The nanopore sequencing device of claim 1, wherein the nanochannel has a path length that is chosen to achieve a desired fluidic, ionic, and/or electrical resistance.

20. The nanopore sequencing device of claim 1, wherein the nanochannel is about 5 nm to about 200 nm wide.

21. The nanopore sequencing device of claim 1, wherein the nanochannel has a footprint with a length of between about 5 μm and about 500 μm.

22. The nanopore sequencing device of claim 21, wherein the path length of the nanochannel is about 1.5 to about 50 times the length of the nanochannel footprint.

23. The nanopore sequencing device of claim 1, further comprising at least one bubble generator, at least one pressure pulse generator, or any combination thereof to control a liquid flow in at least one of the second nanoscale openings.

24. The nanopore sequencing device of any of claim 1, further comprising:
a plurality of middle wells, wherein each middle well is associated with a respective sensing electrode;
each middle well is in fluid communication with the cis well through a respective nanopore; and
each middle well is in fluid communication with the trans well through a respective nanochannel, wherein the respective nanochannel is oriented parallel to the substrate surface.

25. The nanopore sequencing device of claim 24, wherein the respective nanopore is positioned in and through a respective membrane separating each of the middle wells and the cis well.

26. The nanopore sequencing device of claim 24, wherein the trans well is a common trans channel in fluid communication with the plurality of middle wells through respective nanochannels.

27. The nanopore sequencing device of claim 24, wherein the cis well is a common cis channel in fluid communication with the plurality of middle wells through respective nanopores.

28. The nanopore sequencing device of claim 24, wherein the middle wells are arranged in an ordered array.

29. The nanopore sequencing device of claim 24, wherein the device comprises at least 1,000,000 middle wells.

30. The nanopore sequencing device of claim 24, wherein the device further comprises a gas bubble generator configured to generate a gas bubble to modulate or block a flow of current, ions, and/or fluid in the respective nanochannel.

31. The nanopore sequencing device of claim 30, wherein the gas bubble generator comprises the respective sensing electrode configured to generate the gas bubble vis electrolysis.

32. The nanopore sequencing device of claim 30, wherein the gas bubble generator comprises an electrode on the bottom of the nanochannel configured to generate the gas bubble via electrolysis or electrode wetting.

33. The nanopore sequencing device of claim 30, wherein the gas bubble generator comprises a resistive heater underneath the nanochannel configured to generate the gas bubble.

34. The nanopore sequencing device of claim 30, further comprises a gas bubble annihilator.

35. The nanopore sequencing device of claim 34, wherein the gas bubble annihilator comprises an actuator or a piezoelectric element.

36-68. (canceled)
